(19) United States
(12) Patent Application Publication
(10) Pub. No.: US 2026/0297205 A1
(43) Pub. Date: Oct. 1, 2026

(54) GROWTH HORMONE TARGETING POLYPEPTIDES

(71) Applicant: THE UNIVERSITY OF CHICAGO, Chicago, IL (US)

(72) Inventors: Anthony A. KOSSIAKOFF, Chicago, IL (US); Tomasz SLEZAK, Chicago, IL (US)

(73) Assignee: THE UNIVERSITY OF CHICAGO, Chicago, IL (US)

(21) Appl. No.: 19/480,949

(22) PCT Filed: May 3, 2024
(86) PCT No.: PCT/US2024/027698

§ 371 Date: Nov. 3, 2025

Related U.S. Application Data
(60) Provisional application No. 63/499,874, filed on May 3, 2023.

(51) Int. Cl. C07K 16/28; A61K 39/00; A61K 45/06; A61P 35/00; G01N 33/543; G01N 33/575

(52) U.S. Cl. CPC C07K 16/2869; A61K 45/06; A61P 35/00; G01N 33/543; G01N 33/5759; A61K 2039/543; C07K 2317/14; C07K 2317/24; C07K 2317/31; C07K 2317/35; C07K 2317/565; C07K 2317/567; C07K 2317/76; C07K 2317/92; G01N 2333/72

(57) ABSTRACT

Aspects of the present disclosure are directed to growth hormone (GH)-targeting polypeptides, including antibodies, anti-body-drug conjugates, antibody fragments, antibody-like molecules, and chimeric receptors. Also disclosed herein are nucleic acids encoding for such GH-targeting polypeptides and cells comprising such nucleic acids. Described are methods for treatment of acromegaly using GH-targeting polypeptides.

### BACKGROUND

This application claims priority of U.S. Provisional Application No. 63/499,874, filed May 3, 2023, which is hereby incorporated by reference in its entirety.

### INCORPORATION OF SEQUENCE LISTING

The instant application contains a Sequence Listing which has been submitted in XML format and is hereby incorporated by reference in its entirety. Said XML copy, created on May 2, 2024, is named ARCD.P0802WO—Seq Listing.xml and is 247,660 bytes in size.

### I. FIELD OF THE INVENTION

Aspects of the invention relate to at least the fields of molecular biology and medicine.

### II. BACKGROUND

The growth hormone receptor (GHR) is a master regulator of organism development and growth. It is a member of the type I cytokine receptor family and activated specifically and uniquely by growth hormone (GH). The activation of GHR signaling results in changes in cellular metabolism, proliferation, survival, migration and release of cytokines such as IGF1. Consistent with its biological functions, GHR signaling is implicated in many human conditions such as development delays, cancer, aging, longevity, and disorders such as acromegaly.

Acromegaly is a rare disorder with an estimated prevalence of 4 cases per 100,000 people.¹⁰ In the vast majority of cases, the condition is caused by adenomas in the pituitary gland, which lead to an excessive production of human growth hormone (GH) resulting in overproduction of insulin-like growth factor 1 (IGF-1).¹¹ The increased levels of these hormones lead to a spectrum of metabolic, respiratory, cardiovascular comorbidities, and somatic overgrowth, that together result in decreased quality of life and shorten life expectancy. Pegvisomant is a GH variant affinity matured via phage display selection and pegylated at different residues; pegvisomat blocks binding of wildtype hormone to GHR.^{15,16} Pegvisomant presents several disadvantages. First, pegvisomant is a daily injection (20-40 mg) which is difficult for patients to adhere to and represents a reduction in quality of life. Second, some patients develop hepatocellular dysfunctions such as high hepatic transaminase levels in addition to other side effects like lipohypertrophy,¹⁷ suggesting not all patients respond well to this drug. Third, the manufacturing of pevisomat is costly as it requires a second purification step after pegylation.

There exists a need for GH-targeting compositions and methods for use of such compositions in detection of GHR and in treatment of conditions associated with GHR such as acromegaly.

### SUMMARY

To address certain needs in the art, the inventors have generated a diverse number of X-binding proteins, including Fabs and antibodies, that bind to growth hormone (GH). In some aspects, the binding, including at site 2, allows formation of 1:1 complex with the receptor. The binding can block the access for the second receptor binding and thus the dimerization of the GH receptor (GHR). The disclosed potent inhibitory antibodies against GH provide new strategies for therapeutic intervention in conditions like acromegalia, cancer, and longevity. The disclosure describes novel antibody and antigen binding fragments, as well as methods of using these antibodies and fragments. Also described are polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. The disclosure also relates to an antibody comprising at least one polypeptide with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:1-258. Also described are nucleic acids, vectors, or expression vectors encoding antibodies of the disclosure and host cells comprising polypeptides, nucleic acids, vectors, antibodies, or antigen binding fragments of the disclosure. The nucleic acids of the disclosure may be DNA or RNA.

Also described is a method of a making a cell comprising transferring one or more nucleic acid(s) encoding an antibody of the disclosure into a cell. The method may comprise or further comprise culturing the cell under conditions that allow for expression of a polypeptide from the nucleic acid. The method may further comprise isolating the expressed polypeptide. The cell may be further defined as a human cell, B cell, T cell, Chinese hamster ovary, NS0 murine myeloma cell, PER.C6 cell, or a cell described herein.

The disclosure describes a method for treating or preventing acromegaly in a subject, the method comprising administering to the subject an antibody, antigen binding fragment, polypeptide, nucleic acid, or host cell of the disclosure. Also described is a method for evaluating a sample from a subject, the method comprising contacting a biological sample from the subject, or extract thereof, with at least one antibody, antigen binding fragment, polypeptide, composition, or host cell of the disclosure. The antibodies, antigen binding fragments, or compositions of the disclosure may be used to treat a subject having acromegaly.

Methods include a method for treating or preventing cancer in a subject, the method comprising administering to the subject an antibody, antigen binding fragment, polypeptide, nucleic acid, or host cell of the disclosure. Also described is a method for evaluating a sample from a subject, the method comprising contacting a biological sample from the subject, or extract thereof, with at least one antibody, antigen binding fragment, or polypeptide, composition, or host cell of the disclosure. Also disclosed is a method for diagnosing cancer in a subject, the method comprising contacting a biological sample from the subject, or extract thereof, with at least one antibody, antigen binding fragment, composition, or polypeptide of any one of the disclosure. The antibodies, antigen binding fragments, or compositions of the disclosure may be used to treat a subject having cancer.

Provided by the disclosure is an antibody or antigen binding fragment comprising a heavy chain variable region and a light chain variable region, wherein the heavy chain variable region comprises a HCDR1, HCDR2, and HCDR3 from a heavy chain variable region of an antibody clone of Table 1 and wherein the light chain variable region comprises a LCDR1, LCDR2, and LCDR3 from the light chain variable region of the same clone of Table 1. The disclosure also describes an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of Table 1 and wherein the light chain variable region comprises a LCDR1, LCDR2, and LCDR3 having or having at least least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same clone of Table 1. The HCDR1, HCDR2, HCDR3, LCDR1, LCDR2, and/or LCDR3 may be determined from the variable region sequences by methods known in the art. The CDR may be a HCDR1, HCDR2, HCDR3, LCDR1, LCDR2, and/or LCDR3 determined by the Chothia method. The CDR may be a HCDR1, HCDR2, HCDR3, LCDR1, LCDR2, and/or LCDR3 determined by the Kabat method. The CDR may be a HCDR1, HCDR2, HCDR3, LCDR1, LCDR2, and/or LCDR3 determined by the IMGT method.

The disclosure provides for an antibody or antigen binding fragment in which the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. The HCDR1, HCDR2, HCDR2, LCDR1, LCDR2, and LCDR3 may each comprise an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. The HCDR1, HCDR2, HCDR2, LCDR1, LCDR2, and LCDR3 may each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

The disclosure describes an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 from a heavy chain variable region of a antibody clone of Table 1 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 from the light chain variable region of the same antibody clone of Table 1. The antibody or antigen binding fragment may comprise a heavy chain variable region and a light chain variable region, wherein the heavy chain variable region comprises a HCDR1, HCDR2, and HCDR3 having or having at least 80% sequence identity to the HCDR1, HCR2, HCR3 from a heavy chain variable region of a antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1. In some aspects, the heavy chain variable region comprises a HCDR1, HCDR2, and HCDR3 having the amino acid sequence of an of a HCDR1, HCDR2, and HCDR3 of a clone of Table 1 and the light chain variable region comprises a LCDR1, LCDR2, and LCDR3 comprising the amino acid sequence of the LCDR1, LCDR2, and LCDR3 from the light chain variable region of the same clone of Table 1.

The polypeptides of the disclosure may comprise at least two antigen binding fragments, wherein each antigen binding fragment is independently selected from an antigen binding fragment of the disclosure. The polypeptide may be multivalent. The polypeptide may be multispecific. The polypeptide may be bispecific. The polypeptide may comprise, comprise at least, or comprise at most 1, 2, 3, 4, 5, 6, 7, 8, 9, or 10 antigen binding regions. Each antigen binding region may be independently selected from an antigen binding region of the disclosure. The polypeptide may have repeated units of the same antigen binding region, such as at least, at most, or exactly 1, 2, 3, 4, 5, 6, 7, 8, 9, or 10 repeated units.

The heavy chain variable region may comprise an amino acid sequence with at least 80% sequence identity to a heavy chain variable region of an antibody clone of Table 1 and/or the light chain variable region may comprise an amino acid sequence with at least 80% sequence identity to the light chain variable region of the same antibody clone of Table 1. The heavy chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone of Table 1 and/or the light chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone of Table 1. The heavy chain variable region may comprise the amino acid sequence of a heavy chain variable region of an antibody clone of Table 1 and/or the light chain variable region may comprise the amino acid sequence of the same antibody clone of Table 1. The antibody or antigen binding fragment may comprise a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 may comprise an amino acid sequence with at least 80% sequence identity to an HFR1, HFR2, HFR3, and HFR4, respectively, of an antibody clone of Table 1, and the LFR1, LFR2, LFR3, and LFR4 comprises an amino acid sequence with at least 80% sequence identity to the LFR1, LFR2, LFR3, and LFR4, respectively, of the same antibody clone of Table 1. The antibody or antigen binding fragment may comprise a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HFR1, HFR2, HFR3, and HFR4, respectively, of an antibody clone of Table 1, and the LFR1, LFR2, LFR3, and LFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LFR1, LFR2, LFR3, and LFR4, respectively, of the same antibody clone of Table 1. The HFR1, HFR2, HFR3, and HFR4 may comprise the amino acid sequence of an HFR1, HFR2, HFR3, and HFR4, respectively, of an antibody clone of Table 1, and the LFR1, LFR2, LFR3, and LFR4 may comprise the amino acid sequence of the LFR1, LFR2, LFR3, and LFR4, respectively, of the same antibody clone of Table 1. The antibody or antigen binding fragment may comprise a heavy chain and a light chain and wherein the heavy chain comprises an amino acid sequence with at least 70% sequence identity to a heavy chain of an antibody clone of Table 1 and the light chain comprises an amino acid sequence with at least 70% sequence identity to the light chain of the same antibody clone of Table 1. The antibody or antigen binding fragment may comprise a heavy chain and a light chain and wherein the heavy chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain of an antibody clone of Table 1 and the light chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain of the same antibody clone of Table 1. The antibody or antigen binding fragment may comprise a heavy chain and a light chain and wherein the heavy chain comprises the amino acid sequence of an antibody clone of Table 1 and the light chain comprises the amino acid sequence of the same antibody clone of Table 1.

The heavy chain variable region may comprise a heavy chain framework region that has or has at least 80% sequence identity to a heavy chain framework region of an antibody clone of Table 1 and the light chain variable region comprises a light chain framework region that has or has at least 80% sequence identity to a light chain framework region of the same antibody clone of Table 1. The heavy chain variable region may comprise a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of an antibody clone of Table 1 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same antibody clone of Table 1.

The heavy chain variable region may comprise at least 70% sequence identity to the heavy chain variable region of an antibody clone of Table 1 and the light chain variable region comprises at least 70% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 75% sequence identity to the heavy chain variable region of an antibody clone of Table 1 and the light chain variable region comprises at least 75% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 80% sequence identity to the heavy chain variable region of an antibody clone of Table 1 and the light chain variable region comprises at least 80% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 85% sequence identity to the heavy chain variable region of an antibody clone of Table 1 and the light chain variable region comprises at least 85% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 90% sequence identity to the heavy chain variable region of an antibody clone of Table 1 and the light chain variable region comprises at least 90% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 95% sequence identity to the heavy chain variable region of an antibody clone of Table 1 and the light chain variable region comprises at least 95% sequence identity to the light chain variable region of the same antibody clone of Table 1, and wherein the heavy chain and light chain comprise 100% sequence identity to each of the three heavy chain CDRs and three light chain CDRs from the same antibody clone of Table 1.

The antigen binding fragment may be at least 2, 3, 4, 5, or 6 scFv, F(ab′)2, Fab′, Fab, Fv, or rIgG, or combinations thereof. The polypeptide and/or antigen binding fragments of the disclosure may comprise a linker between a heavy chain and light chain variable region or between antigen binding fragments. The linker may be a flexible linker. Exemplary flexible linkers include glycine polymers (G)n, glycine-serine polymers (including, for example, (GS)n, (GSGGS-SEQ ID NO:181)n, (G4S)n and (GGGS-SEQ ID NO:182)n, where n is an integer of at least one. n may be at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG (SEQ ID NO:183), GGSGG (SEQ ID NO:184), GSGSG (SEQ ID NO:185), GSGGG (SEQ ID NO:186), GGGSG (SEQ ID NO:187), GSSSG (SEQ ID NO:188), and the like.

The antibody or antigen binding fragment of the disclosure may be human, chimeric, or humanized. The antibody, or antigen binding fragment may bind GHR with a K_{D} of about 10⁻⁶ M/L or M to about 10⁻¹² M/L or M. The antibody or antigen binding fragment may bind GHR with a K_{D} of about, a K_{D} of at least, or a K_{D} of at most 10, 10⁻¹, 10⁻², 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) M, M/L, μM, nM, or pM. The antibody or antigen binding fragment may bind GHR with a K_{D} of less than 20, 19, 18, 17, 16, 15, 14, 13, 12, 11, 10, 9, 8, 7, 6, 5, 4, 3, 2, or 1 nM, or any range or value derivable therein. The antibody or antigen binding fragment may bind GHR with a K_{D} of less than 2 nM. The antibody or antigen binding fragment may bind GHR with a K_{D} of less than 1 nM. The antibody or antigen binding fragment may bind GHR with a K_{D} of less than 0.5 nM. The antibody or antigen binding fragment may be further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. The antigen binding fragment may be further defined as a single chain variable fragment (scFv), F(ab′)₂, Fab′, Fab, Fv, or rIgG. The antibody, antigen binding fragment, or polypeptide may be operatively linked to a detectable label. Detectable labels are described herein.

Also described herein are multi-specific antibodies and polypeptides. Accordingly, bivalent or bispecific antibodies that comprise two antigen binding fragments, wherein the antigen binding fragment is two of the same antigen binding fragments or two different antigen binding fragments are provided by the description. The disclosure also provides for multi-specific polypeptides. Also described are polypeptides comprising at least 2, 3, 4, 5, or 6 antigen binding fragments. The antigen binding fragment may be at least 2, 3, r, 5, or 6 scFv, F(ab′)₂, Fab′, Fab, Fv, or rIgG, or combinations thereof.

Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure.

In embodiments of the disclosure, it is also contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of any one SEQ ID NOS:1-258 may be excluded.

The subject may be further defined as a human subject or a mammalian subject. The subject may be a laboratory or veterinary animal, such as a pig, horse, cat, cow, rabbit, mouse, rat, or dog. The subject may have one or more symptom of cancer. The subject may not have any symptoms of cancer. The subject may be one that has been diagnosed with cancer. The subject may be one that has not been diagnosed with cancer. The subject may be one that has been previously treated for cancer. The subject may be one that has one or more symptoms of acromegaly. The subject may be one that does not have one or more symptoms of acromegaly. The subject may be one that has been diagnosed with acromegaly. The subject may be one that has not been diagnosed with acromegaly. The subject may be one that has been previously treated for acromegaly. The subject may be administered an additional therapy. The subject may be one that is resistant or has been determined to be resistant to the previous therapy. The additional therapy may comprise one or more of radiotherapy, chemotherapy, and immunotherapy.

The method may further comprise incubating the antibody, antigen binding fragment, or polypeptide under conditions that allow for the binding of the antibody, antigen binding fragment, or polypeptide to antigens in the biological sample or extract thereof. The method may further comprise detecting the binding of an antigen to the antibody, antigen binding fragment, or polypeptide. The method may further comprise contacting the biological sample with at least one capture antibody, antigen, or polypeptide. The at least one capture antibody, antigen binding fragment, or polypeptide may be an antibody, polypeptide, or antigen binding fragment of the disclosure. The capture antibody may be linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. The biological sample may comprise a blood sample, urine sample, fecal sample, or nasopharyngeal sample. The at least one antibody, antigen binding fragment, or polypeptide may be operatively linked to a detectable label. The method may further comprise incubating the antibody, antigen binding fragment, or polypeptide under conditions that allow for the binding of the antibody, antigen binding fragment, or polypeptide to antigens in the biological sample or extract thereof. The method may further comprise detecting the binding of an antigen to the antibody, antigen binding fragment, or polypeptide. The method may further comprise contacting the biological sample with at least one capture antibody, antigen, or polypeptide. The biological sample may comprise a blood sample, urine sample, fecal sample, or nasopharyngeal sample.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1, 2, and 3, respectively and the LCDR1, LCDR2, and LCDR3 comprises an amino acid sequence of SEQ ID NOS:4, 5, and 6, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11, 12, and 13, respectively and the LCDR1, LCDR2, and LCDR3 comprises an amino acid sequence of SEQ ID NOS:4, 5, and 14, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:21, 19, and 20, respectively and the LCDR1, LCDR2, and LCDR3 comprises an amino acid sequence of SEQ ID NOS:4, 5, and 22, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11, 32, and 27, respectively and the LCDR1, LCDR2, and LCDR3 comprises an amino acid sequence of SEQ ID NOS:4, 5, and 28, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:34, 12, and 35, respectively and the LCDR1, LCDR2, and LCDR3 comprises an amino acid sequence of SEQ ID NOS:4, 5, and 36, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:41, 32, and 42, respectively and the LCDR1, LCDR2, and LCDR3 comprises an amino acid sequence of SEQ ID NOS:4, 5, and 43, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:48, 62, and 49, respectively and the LCDR1, LCDR2, and LCDR3 comprises an amino acid sequence of SEQ ID NOS:4, 5, and 50, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:71, 55, and 56, respectively and the LCDR1, LCDR2, and LCDR3 comprises an amino acid sequence of SEQ ID NOS:4, 5, and 57, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:71, 62, and 63, respectively and the LCDR1, LCDR2, and LCDR3 comprises an amino acid sequence of SEQ ID NOS:4, 5, and 64, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:91, 69, and 70, respectively and the LCDR1, LCDR2, and LCDR3 comprises an amino acid sequence of SEQ ID NOS:4, 5, and 72, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:77, 78, and 79, respectively and the LCDR1, LCDR2, and LCDR3 comprises an amino acid sequence of SEQ ID NOS:4, 5, and 106, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:83, 84, and 85, respectively and the LCDR1, LCDR2, and LCDR3 comprises an amino acid sequence of SEQ ID NOS:4, 5, and 86, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:92, 93, and 94, respectively and the LCDR1, LCDR2, and LCDR3 comprises an amino acid sequence of SEQ ID NOS:4, 5, and 106, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:91, 12, and 97, respectively and the LCDR1, LCDR2, and LCDR3 comprises an amino acid sequence of SEQ ID NOS:4, 5, and 98, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11, 62, and 103, respectively and the LCDR1, LCDR2, and LCDR3 comprises an amino acid sequence of SEQ ID NOS:4, 5, and 104, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:111, 113, and 112, respectively and the LCDR1, LCDR2, and LCDR3 comprises an amino acid sequence of SEQ ID NOS:4, 5, and 114, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:119, 32, and 120, respectively and the LCDR1, LCDR2, and LCDR3 comprises an amino acid sequence of SEQ ID NOS:4, 5, and 121, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:127, 62, and 126, respectively and the LCDR1, LCDR2, and LCDR3 comprises an amino acid sequence of SEQ ID NOS:4, 5, and 128, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:133, 62, and 134, respectively and the LCDR1, LCDR2, and LCDR3 comprises an amino acid sequence of SEQ ID NOS:4, 5, and 135, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11, 62, and 140, respectively and the LCDR1, LCDR2, and LCDR3 comprises an amino acid sequence of SEQ ID NOS:4, 5, and 141, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11, 62, and 146, respectively and the LCDR1, LCDR2, and LCDR3 comprises an amino acid sequence of SEQ ID NOS:4, 5, and 147, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:153, 62, and 152, respectively and the LCDR1, LCDR2, and LCDR3 comprises an amino acid sequence of SEQ ID NOS:4, 5, and 154, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11, 62, and 159, respectively and the LCDR1, LCDR2, and LCDR3 comprises an amino acid sequence of SEQ ID NOS:4, 5, and 160, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:21, 32, and 165, respectively and the LCDR1, LCDR2, and LCDR3 comprises an amino acid sequence of SEQ ID NOS:4, 5, and 166, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:172, 171, and 173, respectively and the LCDR1, LCDR2, and LCDR3 comprises an amino acid sequence of SEQ ID NOS:4, 5, and 174, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:179, 180, and 181, respectively and the LCDR1, LCDR2, and LCDR3 comprises an amino acid sequence of SEQ ID NOS:4, 5, and 182, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:92, 187, and 188, respectively and the LCDR1, LCDR2, and LCDR3 comprises an amino acid sequence of SEQ ID NOS:4, 5, and 189, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:127, 62, and 194, respectively and the LCDR1, LCDR2, and LCDR3 comprises an amino acid sequence of SEQ ID NOS:4, 5, and 195, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:127, 62, and 200, respectively and the LCDR1, LCDR2, and LCDR3 comprises an amino acid sequence of SEQ ID NOS:4, 5, and 201, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:206, 113, and 207, respectively and the LCDR1, LCDR2, and LCDR3 comprises an amino acid sequence of SEQ ID NOS:4, 5, and 208, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:213, 62, and 214, respectively and the LCDR1, LCDR2, and LCDR3 comprises an amino acid sequence of SEQ ID NOS:4, 5, and 215, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:220, 221, and 222, respectively and the LCDR1, LCDR2, and LCDR3 comprises an amino acid sequence of SEQ ID NOS:4, 5, and 223, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:228, 229, and 230, respectively and the LCDR1, LCDR2, and LCDR3 comprises an amino acid sequence of SEQ ID NOS:4, 5, and 231, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:48, 113, and 236, respectively and the LCDR1, LCDR2, and LCDR3 comprises an amino acid sequence of SEQ ID NOS:4, 5, and 237, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:172, 242, and 243, respectively and the LCDR1, LCDR2, and LCDR3 comprises an amino acid sequence of SEQ ID NOS:4, 5, and 244, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:92, 249, and 250, respectively and the LCDR1, LCDR2, and LCDR3 comprises an amino acid sequence of SEQ ID NOS:4, 5, and 251, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:153, 78, and 256, respectively and the LCDR1, LCDR2, and LCDR3 comprises an amino acid sequence of SEQ ID NOS:4, 5, and 106, respectively.

The disclosure describes an antibody, antigen binding fragment, or polypeptide comprising a heavy chain variable region and a light chain variable region of SEQ ID NOS:7 and 8, SEQ ID NOS:15 and 16, SEQ ID NOS:23 and 24, SEQ ID NOS:29 and 30, SEQ ID NOS:37 and 38, SEQ ID NOS:44 and 45, SEQ ID NOS:51 and 52, SEQ ID NOS:58 and 59, SEQ ID NOS:65 and 66, SEQ ID NOS:73 and 74, SEQ ID NOS:80 and 108, SEQ ID NOS:87 and 88, SEQ ID NOS:95 and 108, SEQ ID NOS:99 and 100, SEQ ID NOS:105 and 107, SEQ ID NOS:115 and 116, SEQ ID NOS:122 and 123, SEQ ID NOS:129 and 130, SEQ ID NOS:136 and 137, SEQ ID NOS:142 and 143, SEQ ID NOS:148 and 149, SEQ ID NOS:155 and 156, SEQ ID NOS:161 and 162, SEQ ID NOS:167 and 168, SEQ ID NOS:175 and 176, SEQ ID NOS:183 and 184, SEQ ID NOS:190 and 191, SEQ ID NOS:196 and 197 SEQ ID NOS:202 and 203, SEQ ID NOS:209 and 210, SEQ ID NOS:216 and 217, SEQ ID NOS:224 and 225, SEQ ID NOS:232 and 233, SEQ ID NOS:238 and 239, SEQ ID NOS:245 and 246, SEQ ID NOS:252 and 253, or SEQ ID NOS:257 and 108.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments or aspects described herein in the context of the term “comprising” may also be implemented in the context of the term “consisting of” or “consisting essentially of.”

“Individual, “subject,” and “patient” are used interchangeably and can refer to a human or non-human.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Any embodiment or aspect discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. Model GH-mediated signaling inhibition. 1A) GH-mediated signaling inhibition. GHR dimerization upon GH binding activate JAK/STAT signaling pathway. 1B) Model of GH antagonist development. Shared binding site for the GH and generated FABs do not allow GHR dimerization and thus signaling. 1C) Model of GH-mediated signaling inhibition by engineering GH site 2 FABs that would not allow binding to the second GHR and hence dimerization.

FIG. 2. Single-point phage ELISA of the binders selected by phage display. Clones with strong signal against GH were chosen for further sequencing and characterization. Binding of several Fabs is abolished when G120R substitution is introduced into Growth Hormone, suggesting the binding epitope overlapping with GH “site 2” required for Growth Hormone Receptor dimerization and signaling.

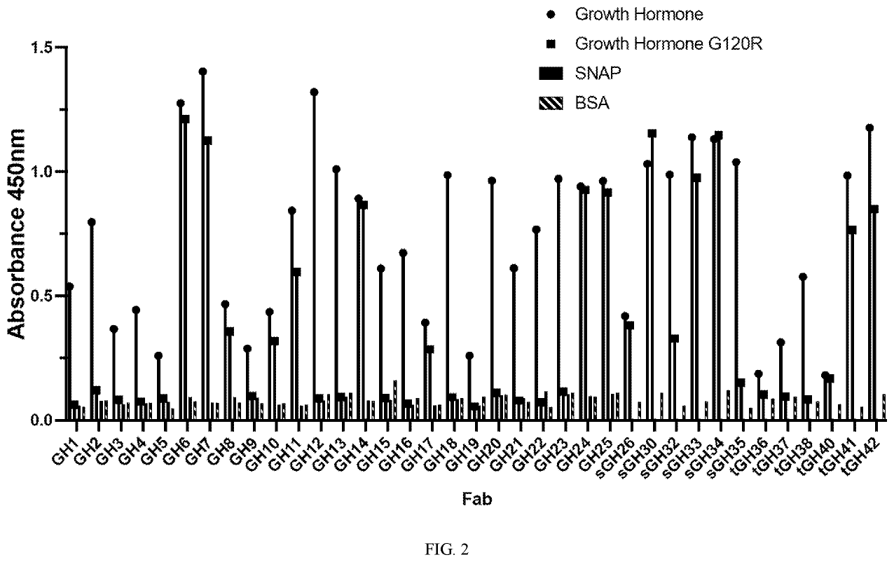

FIG. 3. Characterization of selected Fabs binding against GH. The SPR kinetics shows high-affinity interaction with a very slow dissociation constant. The concentrations of Fab were serially diluted two-fold for each run starting at 50 nM.

FIG. 4. Validation of selected GH Fabs inhibitory activity. pSTAT5 Flow cytometry indicate efficient GH-mediated signaling inhibition upon treatment with selected GH Fabs in vitro. Background pSTAT5 can be observed in the sample indicated as “cells”. B2036 and GHR_TS23 Fabs were used as a positive control. Inhibitory potency of GH Fabs is comparable to GHR_TS23 Fab.

FIG. 5. Specific GH Fabs, when in complex with Growth Hormone, bind to the Growth Hormone Receptor located on the surface of IM9 cells. The binding event was quantified using mean fluorescence intensity (MFI). The complex, introduced at a concentration of 200 nM, was identified using an anti-human Fab antibody labeled with AlexaFluor 647 through flow cytometry. The results are depicted as the mean±standard deviation of three separate experiments (n=3).

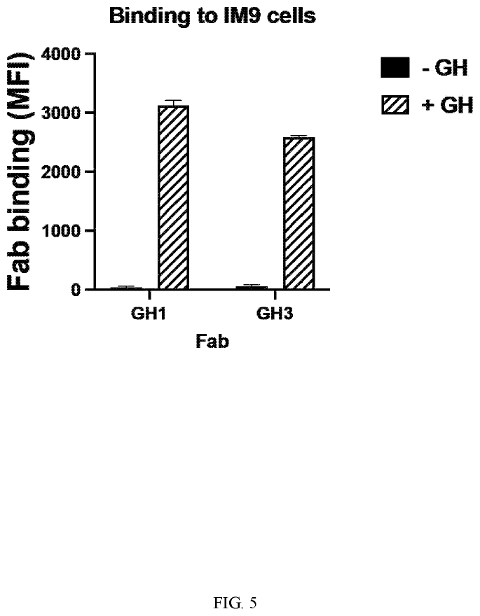

### DETAILED DESCRIPTION OF THE INVENTION

The growth hormone receptor (GHR) belongs to the type I cytokine receptor family and is a master regulator of developmental, cellular, and metabolic processes. Dysregulation of the GHR signaling has been implicated in numerous human diseases including acromegaly, cancer, aging and longevity. Thus, inhibition of GHR signaling may offer therapeutic benefits in many pathological settings. As disclosed herein, antibody phage display was used to generate antibody fragments (FABs) and antibodies (IgGs) that inhibit GHR receptor signaling by binding to GH, including site 2 on GH, which as a result allows formation of 1:1 complex with the receptor however do not grant the access for the second receptor binding and thus the dimerization and downstream signalling. Accordingly, disclosed are compositions comprising various targeting molecules (e.g., antibodies, antibody fragments, antibody-like molecules, antibody-drug conjugates, chimeric antigen receptors, BiTES, etc.) capable of targeting GH, as well as methods for use of such molecules for detection, diagnosis, and treatment of conditions associated with GH, such as acromegaly and cancer.

### I. ANTIBODIES

Aspects of the disclosure relate to antibodies, antigen binding fragments thereof, or polypeptides capable of specifically binding to GH.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (x) and lambda (k). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu ( ), delta (6), gamma (γ), alpha (a), or epsilon (F) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1 (or LCDR1), CDR-L2 (or LCDR2), and CDR-L3 (or LCDR3). The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1 (or HCDR1), CDR-H2 (or HCDR2), and CDR-H3 (or HCDR3). The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to GHR. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab” or “FAB”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fe

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with Antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. ANTIBODY PRODUCTION

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Other methods of making antibodies are through molecular display technologies. These include technologies such as phage display and yeast surface display, have been the major driving force of the development (Winter, 1994; Boder, 2000; Viti, 2000; Bradbury, 2011). These enable display of single-chain Fv (scFv) and Fab fragments on the surface of microorganisms and links the phenotype of a variant to its cognate DNA and thus amino acid sequence (McCafferty, 1990; Kang, 1991; Barbas, 1991). Using these techniques, it is possible to produce a library of an antibody fragment that contains billions of variants and identify variants with a desired binding function from the library in a HTP manner.

In the standard antibody architecture, six surface loops, three each from the heavychain and light-chain variable domains constitute the complementarity determining regions (CDRs). Thus, the antibody and its fragments can be considered as “molecular scaffolds” for presenting CDRs. The sequence diversity of CDRs is the primary source of antibody affinity and specificity. In recombinant antibody libraries, the diversity of CDRs can be either derived from natural immune systems or generated by random mutagenesis, and Fv libraries of both types have been successfully used (Barbas, 1992; Knappik, 2000).

Yeast surface display is another important technical development in antibody engineering. Although the library size achievable with this technique is much smaller than that with phage display, yeast surface display coupled with fluorescence activated cell sorter (FACS) allows for HTP, quantitative characterization of antibody affinity, specificity and expression efficiency (Boder, 2000). The major strengths of phage display include the large size of a library and rapid completion of the selection and propagation cycle (<2 days). Yeast display provides a highly quantitative readout of binding and expression levels using FACS and the ability to precisely define the selection stringency based on equilibrium and kinetic binding parameters as well as expression levels. The integration of these two display methods can provide for an optimal selection of clones that not only are high affinity binders but are stable and express well.

The end products of recombinant antibody screening are the genes encoding Fv and Fab fragments with desired binding characteristics. Bacterial expression systems for Fv and Fab with good yields have been established [Borrebaeck, 1992]. Thus, the phage display clones can be quickly reformatted into an expression cassette for the production of soluble Fv and Fab fragments.

Library design: At least 15-20 residues of CDR residues contribute to antibody binding to its target. Full combinatorial randomization of this many sites together far exceeds the limits of phage and yeast display library diversity. Since the number of sites cannot be significantly reduced, new library strategies have been developed that reduce the 20 natural amino acid genetic code to a smaller subset, while maintaining high functional diversity (Fellouse, 2007). These libraries using a greatly reduced genetic code can produce high affinity Fab and scFv molecules. Although several reduced genetic code libraries have been constructed and tested, the concept has not been optimized for different target classes and especially for the smaller scaffold camelids VHH and FN3 domains (D2). For screening of these novel libraries, we will combine phage display and yeast surface display for optimal efficiency. Humanized antibody scaffolds—In molecular display approaches only the variable regions of the antigen binding loops (CDRs) of the Fab domain are generally diversified. The main architecture of the domain is held constant. This main architecture can be based on a fully human antibody scaffold. This circumvents the need to humanize the scaffold, as is required by methods involving animal immunizations.

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as β-interferon, IL-2, or IL-12, or genes encoding proteins involved in immune helper functions, such as B-7. A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### III. ANTIBODYS, ANTIGEN BINDING FRAGMENTS, AND POLYPEPTIDES

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes an antibody fragment described herein as well as antibody domains, such as HCDR1 (Heavy chain complementarity determining region 1), HCDR2 (Heavy chain complementarity determining region 2), HCDR3 (Heavy chain complementarity determining region 3), LCDR1 (Light chain complementarity determining region 1), LCDR2 (Light chain complementarity determining region 2), LCDR3 (Light chain complementarity determining region 3), HFRW1 (Heavy chain framework region 1), HFRW2 (Heavy chain framework region 2), HFRW3 (Heavy chain framework region 3), HFRW4 (Heavy chain framework region 4), LFRW1 (Light chain framework region 1), LFRW2 (Light chain framework region 21), LFRW3 (Light chain framework region 3), LFRW4 (Light chain framework region 4), VH (Heavy chain variable region), VL (Light chain variable region), CH (Heavy chain constant region), or CL (Light chain constant region).

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NO:1-502.

In some embodiments, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209,210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416,417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:1-258.

In some embodiments, the antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-258.

In some embodiments, the antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-258 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs:1-258.

In some aspects there is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413,414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs:1-258 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222,223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOs:1-258.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 identified in Table 1 and SEQ ID NOs:1-258 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to one of SEQ ID NOs:1-258. In some embodiments, a polypeptide comprises 1, 2, and/or 3 CDRs from one of SEQ ID NOs:1-258. The CDR may be one that has been determined by Kabat, IMGT, or Chothia. In further embodiments, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some embodiments, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some embodiments, the CDRs of SEQ ID NOs:1-258 may further comprise 1, 2, 3, 4, 5, or 6 additional amino acids at the amino or carboxy terminus of the CDR, The additional amino acids may be from the heavy and/or light chain framework regions of SEQ ID NOs:1-258, that are shown as immediately adjacent to the CDRs. Accordingly, embodiments relate to polypeptides comprising an HCDR1 (i.e., CDR-H1), HCDR2 (i.e., CDR-H2), HCDR3(i.e., CDR-H3), LCDR1 (i.e., CDR-L1), LCDR2 (i.e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of Table 1 or SEQ ID NOs:1-258 that are shown as immediately adjacent to the CDRs. Other embodiments relate to antibodies comprising one or more CDRs, wherein the CDR is a fragment of Table 1 or SEQ ID NOs:1-258 and wherein the fragment lacks 1, 2, 3, 4, or 5 amino acids from the amino or carboxy end of the CDR. In some embodiments, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some embodiments, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other embodiments, a polypeptide or protein comprises 1, 2, 3, 4, 5, or 6 CDRs from either or both of the light and heavy variable regions of Table 1 or SEQ ID NOs:1-258, and 1, 2, 3, 4, 5, or 6 CDRs may have 1, 2, and/or 3 amino acid changes with respect to these CDRs. In some embodiments, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### IV. GROWTH HORMONE (GH)

Aspects of the present disclosure are related to compositions and methods for identifying, detecting, and targeting the growth hormone. The growth hormone receptor (GHR; also known as somatotropin receptor) is a master regulator of organism development and growth.^{1, 2} It is a member of the type I cytokine receptor family and is activated specifically and uniquely by growth hormone (GH). Once GH is secreted, it binds to GHR at site 1 in one of the subunits, followed by binding to site 2 in a different subunit using a different protein interface, resulting in receptor dimerization and subsequent conformational change that leads to activation of the associated tyrosine kinase Janus kinase 2 (JAK2) and downstream activation of the signal transducers and activators of transcription (STATs) factors.^{3,4} STAT5 is phosphorylated at several residues which serve as good downstream markers of GHR signaling.⁵ The activation of GHR signaling results in changes in cellular metabolism, proliferation, survival, migration and release of cytokines like IGF1, which at the organisms level resulting in changes growth and development. Consistent with its biological functions GHR signaling is implicated in many human conditions such as development delays, cancer, aging, longevity, and disorders such as acromegaly.⁶⁻⁹

GHR is encoded by the gene GHR. An example mRNA sequence encoding for human GHR is available as NCBI RefSeq NM_000163. An example protein sequence for human GHR is available as NCBI RefSeq NP_000154.

Certain example polypeptide, antibody, and antigen binding fragment sequences are shown below in the following tables.

TABLE 1
Antibody and Antigen Binding Domain Sequences

| | | | SEQ ID |
|---|---|---|---|
| Clone | Description | Sequence | NO: |
| GH1 | HCDR1 | FYYSSI | 1 |
| | HCDR2 | YIYSYSGYTS | 2 |
| | HCDR3 | YSWWGGM | 3 |
| | LCDR1 | SVSSA | 4 |
| | LCDR2 | SASSLYS | 5 |
| | LCDR3 | SYSVPI | 6 |
| | VH | EISEVQLVESGGGLVQPGGSLRLSCAASGFNFYYSSI | 7 |
| | | HWVRQAPGKGLEWVAYIYSYSGYTSYADSVKGRFT | |
| | | ISADTSKNTAYLQMNSLRAEDTAVYYCARYSWWG | |
| | | GMDYWGQGTLVTV | |
| | VL | SDIQMTQSPSSLSASVGDRVTITCRASQSVSSAVAWY | 8 |
| | | QQKPGKAPKLLIYSASSLYSGVPSRFSGSRSGTDFTL | |
| | | TISSLQPEDFATYYCQQSYSVPITFGQGTKVEIKRT | |
| | Full heavy | EISEVQLVESGGGLVQPGGSLRLSCAASGFNFYYSSI | 9 |
| | chain | HWVRQAPGKGLEWVAYIYSYSGYTSYADSVKGRFT | |
| | | ISADTSKNTAYLQMNSLRAEDTAVYYCARYSWWG | |
| | | GMDYWGQGTLVTVSSASTKGPSVFPLAPSSKSTSGG | |
| | | TAALGCLVKDYFPEPVTVSWNSGALTSGVHTFPAVL | |
| | | QSSGLYSLSSVVTVPSSSLGTQTYICNVNHKPSNTKV | |
| | | DKKVEPKSCDKTHT | |
| | Full light | SDIQMTQSPSSLSASVGDRVTITCRASQSVSSAVAWY | 10 |
| | chain | QQKPGKAPKLLIYSASSLYSGVPSRFSGSRSGTDFTL | |
| | | TISSLQPEDFATYYCQQSYSVPITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH2 | HCDR1 | VSSSYI | 11 |
| | HCDR2 | SIYSYYGYTS | 12 |
| | HCDR3 | ENYGNWRSMYSGM | 13 |
| | LCDR1 | SVSSA | 4 |
| | LCDR2 | SASSLYS | 5 |
| | LCDR3 | SYFLPF | 14 |
| | VH | EISEVQLVESGGGLVQPGGSLRLSCAASGFNVSSSYI | 15 |
| | | HWVRQAPGKGLEWVASIYSYYGYTSYADSVKGRFT | |
| | | ISADTSKNTAYLQMNSLRAEDTAVYYCARENYGNW | |
| | | RSMYSGMDYWGQGTLVTV | |
| | VL | SDIQMTQSPSSLSASVGDRVTITCRASQSVSSAVAWY | 16 |
| | | QQKPGKAPKLLIYSASSLYSGVPSRFSGSRSGTDFTL | |
| | | TISSLQPEDFATYYCQQSYFLPFTFGQGTKVEIKRT | |
| | Full heavy | EISEVQLVESGGGLVQPGGSLRLSCAASGFNVSSSYI | 17 |
| | chain | HWVRQAPGKGLEWVASIYSYYGYTSYADSVKGRFT | |
| | | ISADTSKNTAYLQMNSLRAEDTAVYYCARENYGNW | |
| | | RSMYSGMDYWGQGTLVTVSSASTKGPSVFPLAPSSK | |
| | | STSGGTAALGCLVKDYFPEPVTVSWNSGALTSGVHT | |
| | | FPAVLQSSGLYSLSSVVTVPSSSLGTQTYICNVNHKP | |
| | | SNTKVDKKVEPKSCDKTHT | |
| | Full light | SDIQMTQSPSSLSASVGDRVTITCRASQSVSSAVAWY | 18 |
| | chain | QQKPGKAPKLLIYSASSLYSGVPSRFSGSRSGTDFTL | |
| | | TISSLQPEDFATYYCQQSYFLPFTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH3 | HCDR1 | VSSSSI | 21 |
| | HCDR2 | YISSYYGYTS | 19 |
| | HCDR3 | EGWYWWAM | 20 |
| | LCDR1 | SVSSA | 4 |
| | LCDR2 | SASSLYS | 5 |
| | LCDR3 | AWYLPI | 22 |
| | VH | EISEVQLVESGGGLVQPGGSLRLSCAASGFNVSSSSIH | 23 |
| | | WVRQAPGKGLEWVAYISSYYGYTSYADSVKGRFTIS | |
| | | ADTSKNTAYLQMNSLRAEDTAVYYCAREGWYWW | |
| | | AMDYWGQGTLVTV | |
| | VL | SDIQMTQSPSSLSASVGDRVTITCRASQSVSSAVAWY | 24 |
| | | QQKPGKAPKLLIYSASSLYSGVPSRFSGSRSGTDFTL | |
| | | TISSLQPEDFATYYCQQAWYLPITFGQGTKVEIKRT | |
| | Full heavy | EISEVQLVESGGGLVQPGGSLRLSCAASGFNVSSSSIH | 25 |
| | chain | WVRQAPGKGLEWVAYISSYYGYTSYADSVKGRFTIS | |
| | | ADTSKNTAYLQMNSLRAEDTAVYYCAREGWYWW | |
| | | AMDYWGQGTLVTVSSASTKGPSVFPLAPSSKSTSGG | |
| | | TAALGCLVKDYFPEPVTVSWNSGALTSGVHTFPAVL | |
| | | QSSGLYSLSSVVTVPSSSLGTQTYICNVNHKPSNTKV | |
| | | DKKVEPKSCDKTHT | |
| | Full light | SDIQMTQSPSSLSASVGDRVTITCRASQSVSSAVAWY | 26 |
| | chain | QQKPGKAPKLLIYSASSLYSGVPSRFSGSRSGTDFTL | |
| | | TISSLQPEDFATYYCQQAWYLPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH4 | HCDR1 | VSSSYI | 11 |
| | HCDR2 | SIYSSYGYTS | 32 |
| | HCDR3 | ESWGSGYYTWAM | 27 |
| | LCDR1 | SVSSA | 4 |
| | LCDR2 | SASSLYS | 5 |
| | LCDR3 | SWRYPF | 28 |
| | VH | EISEVQLVESGGGLVQPGGSLRLSCAASGFNVSSSYI | 29 |
| | | HWVRQAPGKGLEWVASIYSSYGYTSYADSVKGRFTI | |
| | | SADTSKNTAYLQMNSLRAEDTAVYYCARESWGSGY | |
| | | YTWAMDYWGQGTLVTV | |
| | VL | SDIQMTQSPSSLSASVGDRVTITCRASQSVSSAVAWY | 30 |
| | | QQKPGKAPKLLIYSASSLYSGVPSRFSGSRSGTDFTL | |
| | | TISSLQPEDFATYYCQQSWRYPFTFGQGTKVEIKRT | |
| | Full heavy | EISEVQLVESGGGLVQPGGSLRLSCAASGFNVSSSYI | 31 |
| | chain | HWVRQAPGKGLEWVASIYSSYGYTSYADSVKGRFTI | |
| | | SADTSKNTAYLQMNSLRAEDTAVYYCARESWGSGY | |
| | | YTWAMDYWGQGTLVTVSSASTKGPSVFPLAPSSKS | |
| | | TSGGTAALGCLVKDYFPEPVTVSWNSGALTSGVHTF | |
| | | PAVLQSSGLYSLSSVVTVPSSSLGTQTYICNVNHKPS | |
| | | NTKVDKKVEPKSCDKTHT | |
| | Full light | SDIQMTQSPSSLSASVGDRVTITCRASQSVSSAVAWY | 33 |
| | chain | QQKPGKAPKLLIYSASSLYSGVPSRFSGSRSGTDFTL | |
| | | TISSLQPEDFATYYCQQSWRYPFTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH5 | HCDR1 | VSSYYI | 34 |
| | HCDR2 | SIYSYYGYTS | 12 |
| | HCDR3 | GDNYSWAM | 35 |
| | LCDR1 | SVSSA | 4 |
| | LCDR2 | SASSLYS | 5 |
| | LCDR3 | SWQYPF | 36 |
| | VH | EISEVQLVESGGGLVQPGGSLRLSCAASGFNVSSYYI | 37 |
| | | HWVRQAPGKGLEWVASIYSYYGYTSYADSVKGRFT | |
| | | ISADTSKNTAYLQMNSLRAEDTAVYYCARGDNYSW | |
| | | AMDYWGQGTLVTV | |
| | VL | SDIQMTQSPSSLSASVGDRVTITCRASQSVSSAVAWY | 38 |
| | | QQKPGKAPKLLIYSASSLYSGVPSRFSGSRSGTDFTL | |
| | | TISSLQPEDFATYYCQQSWQYPFTFGQGTKVEIKRT | |
| | Full heavy | EISEVQLVESGGGLVQPGGSLRLSCAASGFNVSSYYI | 39 |
| | chain | HWVRQAPGKGLEWVASIYSYYGYTSYADSVKGRFT | |
| | | ISADTSKNTAYLQMNSLRAEDTAVYYCARGDNYSW | |
| | | AMDYWGQGTLVTVSSASTKGPSVFPLAPSSKSTSGG | |
| | | TAALGCLVKDYFPEPVTVSWNSGALTSGVHTFPAVL | |
| | | QSSGLYSLSSVVTVPSSSLGTQTYICNVNHKPSNTKV | |
| | | DKKVEPKSCDKTHT | |
| | Full light | SDIQMTQSPSSLSASVGDRVTITCRASQSVSSAVAWY | 40 |
| | chain | QQKPGKAPKLLIYSASSLYSGVPSRFSGSRSGTDFTL | |
| | | TISSLQPEDFATYYCQQSWQYPFTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH6 | HCDR1 | VSYSSI | 41 |
| | HCDR2 | SIYSSYGYTS | 32 |
| | HCDR3 | GHGSMWAM | 42 |
| | LCDR1 | SVSSA | 4 |
| | LCDR2 | SASSLYS | 5 |
| | LCDR3 | SWSLPF | 43 |
| | VH | EISEVQLVESGGGLVQPGGSLRLSCAASGFNVSYSSI | 44 |
| | | HWVRQAPGKGLEWVASIYSSYGYTSYADSVKGRFTI | |
| | | SADTSKNTAYLQMNSLRAEDTAVYYCARGHGSMW | |
| | | AMDYWGQGTLVTV | |
| | VL | SDIQMTQSPSSLSASVGDRVTITCRASQSVSSAVAWY | 45 |
| | | QQKPGKAPKLLIYSASSLYSGVPSRFSGSRSGTDFTL | |
| | | TISSLQPEDFATYYCQQSWSLPFTFGQGTKVEIKRT | |
| | Full heavy | EISEVQLVESGGGLVQPGGSLRLSCAASGFNVSYSSI | 46 |
| | chain | HWVRQAPGKGLEWVASIYSSYGYTSYADSVKGRFTI | |
| | | SADTSKNTAYLQMNSLRAEDTAVYYCARGHGSMW | |
| | | AMDYWGQGTLVTVSSASTKGPSVFPLAPSSKSTSGG | |
| | | TAALGCLVKDYFPEPVTVSWNSGALTSGVHTFPAVL | |
| | | QSSGLYSLSSVVTVPSSSLGTQTYICNVNHKPSNTKV | |
| | | DKKVEPKSCDKTHT | |
| | Full light | SDIQMTQSPSSLSASVGDRVTITCRASQSVSSAVAWY | 47 |
| | chain | QQKPGKAPKLLIYSASSLYSGVPSRFSGSRSGTDFTL | |
| | | TISSLQPEDFATYYCQQSWSLPFTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH7 | HCDR1 | VYSSSI | 48 |
| | HCDR2 | SIYSYSGYTS | 62 |
| | HCDR3 | GRYSWFAL | 49 |
| | LCDR1 | SVSSA | 4 |
| | LCDR2 | SASSLYS | 5 |
| | LCDR3 | YWSFPI | 50 |
| | VH | EISEVQLVESGGGLVQPGGSLRLSCAASGFNVYSSSI | 51 |
| | | HWVRQAPGKGLEWVASIYSYSGYTSYADSVKGRFTI | |
| | | SADTSKNTAYLQMNSLRAEDTAVYYCARGRYSWFA | |
| | | LDYWGQGTLVTV | |
| | VL | SDIQMTQSPSSLSASVGDRVTITCRASQSVSSAVAWY | 52 |
| | | QQKPGKAPKLLIYSASSLYSGVPSRFSGSRSGTDFTL | |
| | | TISSLQPEDFATYYCQQYWSFPITFGQGTKVEIKRT | |
| | Full heavy | EISEVQLVESGGGLVQPGGSLRLSCAASGFNVYSSSI | 53 |
| | chain | HWVRQAPGKGLEWVASIYSYSGYTSYADSVKGRFTI | |
| | | SADTSKNTAYLQMNSLRAEDTAVYYCARGRYSWFA | |
| | | LDYWGQGTLVTVSSASTKGPSVFPLAPSSKSTSGGT | |
| | | AALGCLVKDYFPEPVTVSWNSGALTSGVHTFPAVLQ | |
| | | SSGLYSLSSVVTVPSSSLGTQTYICNVNHKPSNTKVD | |
| | | KKVEPKSCDKTHT | |
| | Full light | SDIQMTQSPSSLSASVGDRVTITCRASQSVSSAVAWY | 54 |
| | chain | QQKPGKAPKLLIYSASSLYSGVPSRFSGSRSGTDFTL | |
| | | TISSLQPEDFATYYCQQYWSFPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH8 | HCDR1 | VYSSYI | 71 |
| | HCDR2 | SISSSYGYTS | 55 |
| | HCDR3 | GYYYSYAM | 56 |
| | LCDR1 | SVSSA | 4 |
| | LCDR2 | SASSLYS | 5 |
| | LCDR3 | SWSQPF | 57 |
| | VH | EISEVQLVESGGGLVQPGGSLRLSCAASGFNVYSSYI | 58 |
| | | HWVRQAPGKGLEWVASISSSYGYTSYADSVKGRFTI | |
| | | SADTSKNTAYLQMNSLRAEDTAVYYCARGYYYSYA | |
| | | MDYWGQGTLVTV | |
| | VL | SDIQMTQSPSSLSASVGDRVTITCRASQSVSSAVAWY | 59 |
| | | QQKPGKAPKLLIYSASSLYSGVPSRFSGSRSGTDFTL | |
| | | TISSLQPEDFATYYCQQSWSQPFTFGQGTKVEIKRT | |
| | Full heavy | EISEVQLVESGGGLVQPGGSLRLSCAASGFNVYSSYI | 60 |
| | chain | HWVRQAPGKGLEWVASISSSYGYTSYADSVKGRFTI | |
| | | SADTSKNTAYLQMNSLRAEDTAVYYCARGYYYSYA | |
| | | MDYWGQGTLVTVSSASTKGPSVFPLAPSSKSTSGGT | |
| | | AALGCLVKDYFPEPVTVSWNSGALTSGVHTFPAVLQ | |
| | | SSGLYSLSSVVTVPSSSLGTQTYICNVNHKPSNTKVD | |
| | | KKVEPKSCDKTHT | |
| | Full light | SDIQMTQSPSSLSASVGDRVTITCRASQSVSSAVAWY | 61 |
| | chain | QQKPGKAPKLLIYSASSLYSGVPSRFSGSRSGTDFTL | |
| | | TISSLQPEDFATYYCQQSWSQPFTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH9 | HCDR1 | VYSSYI | 71 |
| | HCDR2 | SIYSYSGYTS | 62 |
| | HCDR3 | HSSWAM | 63 |
| | LCDR1 | SVSSA | 4 |
| | LCDR2 | SASSLYS | 5 |
| | LCDR3 | SSFVPL | 64 |
| | VH | EISEVQLVESGGGLVQPGGSLRLSCAASGFNVYSSYI | 65 |
| | | HWVRQAPGKGLEWVASIYSYSGYTSYADSVKGRFTI | |
| | | SADTSKNTAYLQMNSLRAEDTAVYYCARHSSWAM | |
| | | DYWGQGTLVTV | |
| | VL | SDIQMTQSPSSLSASVGDRVTITCRASQSVSSAVAWY | 66 |
| | | QQKPGKAPKLLIYSASSLYSGVPSRFSGSRSGTDFTL | |
| | | TISSLQPEDFATYYCQQSSFVPLTFGQGTKVEIKRT | |
| | Full heavy | EISEVQLVESGGGLVQPGGSLRLSCAASGFNVYSSYI | 67 |
| | chain | HWVRQAPGKGLEWVASIYSYSGYTSYADSVKGRFTI | |
| | | SADTSKNTAYLQMNSLRAEDTAVYYCARHSSWAM | |
| | | DYWGQGTLVTVSSASTKGPSVFPLAPSSKSTSGGTA | |
| | | ALGCLVKDYFPEPVTVSWNSGALTSGVHTFPAVLQS | |
| | | SGLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 68 |
| | chain | QQKPGKAPKLLIYSASSLYSGVPSRFSGSRSGTDFTL | |
| | | TISSLQPEDFATYYCQQSSFVPLTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH10 | HCDR1 | FYSSSI | 91 |
| | HCDR2 | YISPYSGYTS | 69 |
| | HCDR3 | IAYKWYMPYYYGM | 70 |
| | LCDR1 | SVSSA | 4 |
| | LCDR2 | SASSLYS | 5 |
| | LCDR3 | SLSSLV | 72 |
| | VH | EISEVQLVESGGGLVQPGGSLRLSCAASGFNFYSSSIH | 73 |
| | | WVRQAPGKGLEWVAYISPYSGYTSYADSVKGRFTIS | |
| | | ADTSKNTAYLQMNSLRAEDTAVYYCARIAYKWYM | |
| | | PYYYGMDYWGQGTLVTV | |
| | VL | SDIQMTQSPSSLSASVGDRVTITCRASQSVSSAVAWY | 74 |
| | | QQKPGKAPKLLIYSASSLYSGVPSRFSGSRSGTDFTL | |
| | | TISSLQPEDFATYYCQQSLSSLVTFGQGTKVEIKRT | |
| | Full heavy | EISEVQLVESGGGLVQPGGSLRLSCAASGFNFYSSSIH | 75 |
| | chain | WVRQAPGKGLEWVAYISPYSGYTSYADSVKGRFTIS | |
| | | ADTSKNTAYLQMNSLRAEDTAVYYCARIAYKWYM | |
| | | PYYYGMDYWGQGTLVTVSSASTKGPSVFPLAPSSKS | |
| | | TSGGTAALGCLVKDYFPEPVTVSWNSGALTSGVHTF | |
| | | PAVLQSSGLYSLSSVVTVPSSSLGTQTYICNVNHKPS | |
| | | NTKVDKKVEPKSCDKTHT | |
| | Full light | SDIQMTQSPSSLSASVGDRVTITCRASQSVSSAVAWY | 76 |
| | chain | QQKPGKAPKLLIYSASSLYSGVPSRFSGSRSGTDFTL | |
| | | TISSLQPEDFATYYCQQSLSSLVTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH11 | HCDR1 | IYSSSI | 77 |
| | HCDR2 | SISSSSGSTS | 78 |
| | HCDR3 | KYSGYYSNWYYPAL | 79 |
| | LCDR1 | SVSSA | 4 |
| | LCDR2 | SASSLYS | 5 |
| | LCDR3 | SSSSLI | 106 |
| | VH | EISEVQLVESGGGLVQPGGSLRLSCAASGFNIYSSSIH | 80 |
| | | WVRQAPGKGLEWVASISSSSGSTSYADSVKGRFTIS | |
| | | ADTSKNTAYLQMNSLRAEDTAVYYCARKYSGYYSN | |
| | | WYYPALDYWGQGTLVTV | |
| | VL | SDIQMTQSPSSLSASVGDRVTITCRASQSVSSAVAWY | 108 |
| | | QQKPGKAPKLLIYSASSLYSGVPSRFSGSRSGTDFTL | |
| | | TISSLQPEDFATYYCQQSSSSLITFGQGTKVEIKRT | |
| | Full heavy | EISEVQLVESGGGLVQPGGSLRLSCAASGFNIYSSSIH | 81 |
| | chain | WVRQAPGKGLEWVASISSSSGSTSYADSVKGRFTIS | |
| | | ADTSKNTAYLQMNSLRAEDTAVYYCARKYSGYYSN | |
| | | WYYPALDYWGQGTLVTVSSASTKGPSVFPLAPSSKS | |
| | | TSGGTAALGCLVKDYFPEPVTVSWNSGALTSGVHTF | |
| | | PAVLQSSGLYSLSSVVTVPSSSLGTQTYICNVNHKPS | |
| | | NTKVDKKVEPKSCDKTHT | |
| | Full light | SDIQMTQSPSSLSASVGDRVTITCRASQSVSSAVAWY | 82 |
| | chain | QQKPGKAPKLLIYSASSLYSGVPSRFSGSRSGTDFTL | |
| | | TISSLQPEDFATYYCQQSSSSLITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH12 | HCDR1 | LSSSYI | 83 |
| | HCDR2 | SIYPYSGYTS | 84 |
| | HCDR3 | MYWTGAL | 85 |
| | LCDR1 | SVSSA | 4 |
| | LCDR2 | SASSLYS | 5 |
| | LCDR3 | SSYSPI | 86 |
| | VH | EISEVQLVESGGGLVQPGGSLRLSCAASGFNLSSSYI | 87 |
| | | HWVRQAPGKGLEWVASIYPYSGYTSYADSVKGRFTI | |
| | | SADTSKNTAYLQMNSLRAEDTAVYYCARMYWTGA | |
| | | LDYWGQGTLVTV | |
| | VL | SDIQMTQSPSSLSASVGDRVTITCRASQSVSSAVAWY | 88 |
| | | QQKPGKAPKLLIYSASSLYSGVPSRFSGSRSGTDFTL | |
| | | TISSLQPEDFATYYCQQSSYSPITFGQGTKVEIKRT | |
| | Full heavy | EISEVQLVESGGGLVQPGGSLRLSCAASGFNLSSSYI | 89 |
| | chain | HWVRQAPGKGLEWVASIYPYSGYTSYADSVKGRFTI | |
| | | SADTSKNTAYLQMNSLRAEDTAVYYCARMYWTGA | |
| | | LDYWGQGTLVTVSSASTKGPSVFPLAPSSKSTSGGT | |
| | | AALGCLVKDYFPEPVTVSWNSGALTSGVHTFPAVLQ | |
| | | SSGLYSLSSVVTVPSSSLGTQTYICNVNHKPSNTKVD | |
| | | KKVEPKSCDKTHT | |
| | Full light | SDIQMTQSPSSLSASVGDRVTITCRASQSVSSAVAWY | 90 |
| | chain | QQKPGKAPKLLIYSASSLYSGVPSRFSGSRSGTDFTL | |
| | | TISSLQPEDFATYYCQQSSYSPITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH13 | HCDR1 | FSSSSI | 92 |
| | HCDR2 | SISSYYSSTY | 93 |
| | HCDR3 | QYTSPWHWYKAM | 94 |
| | LCDR1 | SVSSA | 4 |
| | LCDR2 | SASSLYS | 5 |
| | LCDR3 | SSSSLI | 106 |
| | VH | EISEVQLVESGGGLVQPGGSLRLSCAASGFNFSSSSIH | 95 |
| | | WVRQAPGKGLEWVASISSYYSSTYYADSVKGRFTIS | |
| | | ADTSKNTAYLQMNSLRAEDTAVYYCARQYTSPWH | |
| | | WYKAMDYWGQGTLVTV | |
| | VL | SDIQMTQSPSSLSASVGDRVTITCRASQSVSSAVAWY | 108 |
| | | QQKPGKAPKLLIYSASSLYSGVPSRFSGSRSGTDFTL | |
| | | TISSLQPEDFATYYCQQSSSSLITFGQGTKVEIKRT | |
| | Full heavy | EISEVQLVESGGGLVQPGGSLRLSCAASGFNFSSSSIH | 96 |
| | chain | WVRQAPGKGLEWVASISSYYSSTYYADSVKGRFTIS | |
| | | ADTSKNTAYLQMNSLRAEDTAVYYCARQYTSPWH | |
| | | WYKAMDYWGQGTLVTVSSASTKGPSVFPLAPSSKS | |
| | | TSGGTAALGCLVKDYFPEPVTVSWNSGALTSGVHTF | |
| | | PAVLQSSGLYSLSSVVTVPSSSLGTQTYICNVNHKPS | |
| | | NTKVDKKVEPKSCDKTHT | |
| | Full light | SDIQMTQSPSSLSASVGDRVTITCRASQSVSSAVAWY | 82 |
| | chain | QQKPGKAPKLLIYSASSLYSGVPSRFSGSRSGTDFTL | |
| | | TISSLQPEDFATYYCQQSSSSLITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH14 | HCDR1 | FYSSSI | 91 |
| | HCDR2 | SIYSYYGYTS | 12 |
| | HCDR3 | SGGYIGF | 97 |
| | LCDR1 | SVSSA | 4 |
| | LCDR2 | SASSLYS | 5 |
| | LCDR3 | YWEYPF | 98 |
| | VH | EISEVQLVESGGGLVQPGGSLRLSCAASGFNFYSSSIH | 99 |
| | | WVRQAPGKGLEWVASIYSYYGYTSYADSVKGRFTIS | |
| | | ADTSKNTAYLQMNSLRAEDTAVYYCARSGGYIGFD | |
| | | YWGQGTLVTV | |
| | VL | SDIQMTQSPSSLSASVGDRVTITCRASQSVSSAVAWY | 100 |
| | | QQKPGKAPKLLIYSASSLYSGVPSRFSGSRSGTDFTL | |
| | | TISSLQPEDFATYYCQQYWEYPFTFGQGTKVEIKRT | |
| | Full heavy | EISEVQLVESGGGLVQPGGSLRLSCAASGFNFYSSSIH | 101 |
| | chain | WVRQAPGKGLEWVASIYSYYGYTSYADSVKGRFTIS | |
| | | ADTSKNTAYLQMNSLRAEDTAVYYCARSGGYIGFD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 102 |
| | chain | QQKPGKAPKLLIYSASSLYSGVPSRFSGSRSGTDFTL | |
| | | TISSLQPEDFATYYCQQYWEYPFTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH15 | HCDR1 | VSSSYI | 11 |
| | HCDR2 | SIYSYSGYTS | 62 |
| | HCDR3 | TSYGRGAL | 103 |
| | LCDR1 | SVSSA | 4 |
| | LCDR2 | SASSLYS | 5 |
| | LCDR3 | WSWYPI | 104 |
| | VH | EISEVQLVESGGGLVQPGGSLRLSCAASGFNVSSSYI | 105 |
| | | HWVRQAPGKGLEWVASIYSYSGYTSYADSVKGRFTI | |
| | | SADTSKNTAYLQMNSLRAEDTAVYYCARTSYGRGA | |
| | | LDYWGQGTLVTV | |
| | VL | SDIQMTQSPSSLSASVGDRVTITCRASQSVSSAVAWY | 107 |
| | | QQKPGKAPKLLIYSASSLYSGVPSRFSGSRSGTDFTL | |
| | | TISSLQPEDFATYYCQQWSWYPITFGQGTKVEIKRT | |
| | Full heavy | EISEVQLVESGGGLVQPGGSLRLSCAASGFNVSSSYI | 109 |
| | chain | HWVRQAPGKGLEWVASIYSYSGYTSYADSVKGRFTI | |
| | | SADTSKNTAYLQMNSLRAEDTAVYYCARTSYGRGA | |
| | | LDYWGQGTLVTVSSASTKGPSVFPLAPSSKSTSGGT | |
| | | AALGCLVKDYFPEPVTVSWNSGALTSGVHTFPAVLQ | |
| | | SSGLYSLSSVVTVPSSSLGTQTYICNVNHKPSNTKVD | |
| | | KKVEPKSCDKTHT | |
| | Full light | SDIQMTQSPSSLSASVGDRVTITCRASQSVSSAVAWY | 110 |
| | chain | QQKPGKAPKLLIYSASSLYSGVPSRFSGSRSGTDFTL | |
| | | TISSLQPEDFATYYCQQWSWYPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH16 | HCDR1 | LSSYYI | 111 |
| | HCDR2 | SIYPSYGYTS | 113 |
| | HCDR3 | WGKWYGYPFSGL | 112 |
| | LCDR1 | SVSSA | 4 |
| | LCDR2 | SASSLYS | 5 |
| | LCDR3 | DWYFPI | 114 |
| | VH | EISEVQLVESGGGLVQPGGSLRLSCAASGFNLSSYYI | 115 |
| | | HWVRQAPGKGLEWVASIYPSYGYTSYADSVKGRFTI | |
| | | SADTSKNTAYLQMNSLRAEDTAVYYCARWGKWYG | |
| | | YPFSGLDYWGQGTLVTV | |
| | VL | SDIQMTQSPSSLSASVGDRVTITCRASQSVSSAVAWY | 116 |
| | | QQKPGKAPKLLIYSASSLYSGVPSRFSGSRSGTDFTL | |
| | | TISSLQPEDFATYYCQQDWYFPITFGQGTKVEIKRT | |
| | Full heavy | EISEVQLVESGGGLVQPGGSLRLSCAASGFNLSSYYI | 117 |
| | chain | HWVRQAPGKGLEWVASIYPSYGYTSYADSVKGRFTI | |
| | | SADTSKNTAYLQMNSLRAEDTAVYYCARWGKWYG | |
| | | YPFSGLDYWGQGTLVTVSSASTKGPSVFPLAPSSKST | |
| | | SGGTAALGCLVKDYFPEPVTVSWNSGALTSGVHTFP | |
| | | AVLQSSGLYSLSSVVTVPSSSLGTQTYICNVNHKPSN | |
| | | TKVDKKVEPKSCDKTHT | |
| | Full light | SDIQMTQSPSSLSASVGDRVTITCRASQSVSSAVAWY | 118 |
| | chain | QQKPGKAPKLLIYSASSLYSGVPSRFSGSRSGTDFTL | |
| | | TISSLQPEDFATYYCQQDWYFPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH17 | HCDR1 | ISSI | 119 |
| | HCDR2 | SIYSSYGYTS | 32 |
| | HCDR3 | WLYEAM | 120 |
| | LCDR1 | SVSSA | 4 |
| | LCDR2 | SASSLYS | 5 |
| | LCDR3 | FWFFPI | 121 |
| | VH | EISEVQLVESGGGLVQPGGSLRLSCAASGFNISSIHW | 122 |
| | | VRQAPGKGLEWVASIYSSYGYTSYADSVKGRFTISA | |
| | | DTSKNTAYLQMNSLRAEDTAVYYCARWLYEAMDY | |
| | | WGQGTLVTV | |
| | VL | SDIQMTQSPSSLSASVGDRVTITCRASQSVSSAVAWY | 123 |
| | | QQKPGKAPKLLIYSASSLYSGVPSRFSGSRSGTDFTL | |
| | | TISSLQPEDFATYYCQQFWFFPITFGQGTKVEIKRT | |
| | Full heavy | EISEVQLVESGGGLVQPGGSLRLSCAASGFNISSIHW | 124 |
| | chain | VRQAPGKGLEWVASIYSSYGYTSYADSVKGRFTISA | |
| | | DTSKNTAYLQMNSLRAEDTAVYYCARWLYEAMDY | |
| | | WGQGTLVTVSSASTKGPSVFPLAPSSKSTSGGTAAL | |
| | | GCLVKDYFPEPVTVSWNSGALTSGVHTFPAVLQSSG | |
| | | LYSLSSVVTVPSSSLGTQTYICNVNHKPSNTKVDKK | |
| | | VEPKSCDKTHT | |
| | Full light | SDIQMTQSPSSLSASVGDRVTITCRASQSVSSAVAWY | 125 |
| | chain | QQKPGKAPKLLIYSASSLYSGVPSRFSGSRSGTDFTL | |
| | | TISSLQPEDFATYYCQQFWFFPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH18 | HCDR1 | FYSSYI | 127 |
| | HCDR2 | SIYSYSGYTS | 62 |
| | HCDR3 | YKEPFGM | 126 |
| | LCDR1 | SVSSA | 4 |
| | LCDR2 | SASSLYS | 5 |
| | LCDR3 | SNYMPL | 128 |
| | VH | EISEVQLVESGGGLVQPGGSLRLSCAASGFNFYSSYI | 129 |
| | | HWVRQAPGKGLEWVASIYSYSGYTSYADSVKGRFTI | |
| | | SADTSKNTAYLQMNSLRAEDTAVYYCARYKEPFGM | |
| | | DYWGQGTLVTV | |
| | VL | SDIQMTQSPSSLSASVGDRVTITCRASQSVSSAVAWY | 130 |
| | | QQKPGKAPKLLIYSASSLYSGVPSRFSGSRSGTDFTL | |
| | | TISSLQPEDFATYYCQQSNYMPLTFGQGTKVEIKRT | |
| | Full heavy | EISEVQLVESGGGLVQPGGSLRLSCAASGFNFYSSYI | 131 |
| | chain | HWVRQAPGKGLEWVASIYSYSGYTSYADSVKGRFTI | |
| | | SADTSKNTAYLQMNSLRAEDTAVYYCARYKEPFGM | |
| | | DYWGQGTLVTVSSASTKGPSVFPLAPSSKSTSGGTA | |
| | | ALGCLVKDYFPEPVTVSWNSGALTSGVHTFPAVLQS | |
| | | SGLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 132 |
| | chain | QQKPGKAPKLLIYSASSLYSGVPSRFSGSRSGTDFTL | |
| | | TISSLQPEDFATYYCQQSNYMPLTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH19 | HCDR1 | FSYSYI | 133 |
| | HCDR2 | SIYSYSGYTS | 62 |
| | HCDR3 | YKWWAM | 134 |
| | LCDR1 | SVSSA | 4 |
| | LCDR2 | SASSLYS | 5 |
| | LCDR3 | SGSWPI | 135 |
| | VH | EISEVQLVESGGGLVQPGGSLRLSCAASGFNFSYSYI | 136 |
| | | HWVRQAPGKGLEWVASIYSYSGYTSYADSVKGRFTI | |
| | | SADTSKNTAYLQMNSLRAEDTAVYYCARYKWWA | |
| | | MDYWGQGTLVTV | |
| | VL | SDIQMTQSPSSLSASVGDRVTITCRASQSVSSAVAWY | 137 |
| | | QQKPGKAPKLLIYSASSLYSGVPSRFSGSRSGTDFTL | |
| | | TISSLQPEDFATYYCQQSGSWPITFGQGTKVEIKRT | |
| | Full heavy | EISEVQLVESGGGLVQPGGSLRLSCAASGFNFSYSYI | 138 |
| | chain | HWVRQAPGKGLEWVASIYSYSGYTSYADSVKGRFTI | |
| | | SADTSKNTAYLQMNSLRAEDTAVYYCARYKWWA | |
| | | MDYWGQGTLVTVSSASTKGPSVFPLAPSSKSTSGGT | |
| | | AALGCLVKDYFPEPVTVSWNSGALTSGVHTFPAVLQ | |
| | | SSGLYSLSSVVTVPSSSLGTQTYICNVNHKPSNTKVD | |
| | | KKVEPKSCDKTHT | |
| | Full light | SDIQMTQSPSSLSASVGDRVTITCRASQSVSSAVAWY | 139 |
| | chain | QQKPGKAPKLLIYSASSLYSGVPSRFSGSRSGTDFTL | |
| | | TISSLQPEDFATYYCQQSGSWPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH20 | HCDR1 | VSSSYI | 11 |
| | HCDR2 | SIYSYSGYTS | 62 |
| | HCDR3 | YKWYAM | 140 |
| | LCDR1 | SVSSA | 4 |
| | LCDR2 | SASSLYS | 5 |
| | LCDR3 | SSWYPI | 141 |
| | VH | EISEVQLVESGGGLVQPGGSLRLSCAASGFNVSSSYI | 142 |
| | | HWVRQAPGKGLEWVASIYSYSGYTSYADSVKGRFTI | |
| | | SADTSKNTAYLQMNSLRAEDTAVYYCARYKWYAM | |
| | | DYWGQGTLVTV | |
| | VL | SDIQMTQSPSSLSASVGDRVTITCRASQSVSSAVAWY | 143 |
| | | QQKPGKAPKLLIYSASSLYSGVPSRFSGSRSGTDFTL | |
| | | TISSLQPEDFATYYCQQSSWYPITFGQGTKVEIKRT | |
| | Full heavy | EISEVQLVESGGGLVQPGGSLRLSCAASGFNVSSSYI | 144 |
| | chain | HWVRQAPGKGLEWVASIYSYSGYTSYADSVKGRFTI | |
| | | SADTSKNTAYLQMNSLRAEDTAVYYCARYKWYAM | |
| | | DYWGQGTLVTVSSASTKGPSVFPLAPSSKSTSGGTA | |
| | | ALGCLVKDYFPEPVTVSWNSGALTSGVHTFPAVLQS | |
| | | SGLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 145 |
| | chain | QQKPGKAPKLLIYSASSLYSGVPSRFSGSRSGTDFTL | |
| | | TISSLQPEDFATYYCQQSSWYPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH21 | HCDR1 | VSSSYI | 11 |
| | HCDR2 | SIYSYSGYTS | 62 |
| | HCDR3 | YMYYAM | 146 |
| | LCDR1 | SVSSA | 4 |
| | LCDR2 | SASSLYS | 5 |
| | LCDR3 | SSWSPI | 147 |
| | VH | EISEVQLVESGGGLVQPGGSLRLSCAASGFNVSSSYI | 148 |
| | | HWVRQAPGKGLEWVASIYSYSGYTSYADSVKGRFTI | |
| | | SADTSKNTAYLQMNSLRAEDTAVYYCARYMYYAM | |
| | | DYWGQGTLVTV | |
| | VL | SDIQMTQSPSSLSASVGDRVTITCRASQSVSSAVAWY | 149 |
| | | QQKPGKAPKLLIYSASSLYSGVPSRFSGSRSGTDFTL | |
| | | TISSLQPEDFATYYCQQSSWSPITFGQGTKVEIKRT | |
| | Full heavy | EISEVQLVESGGGLVQPGGSLRLSCAASGFNVSSSYI | 150 |
| | chain | HWVRQAPGKGLEWVASIYSYSGYTSYADSVKGRFTI | |
| | | SADTSKNTAYLQMNSLRAEDTAVYYCARYMYYAM | |
| | | DYWGQGTLVTVSSASTKGPSVFPLAPSSKSTSGGTA | |
| | | ALGCLVKDYFPEPVTVSWNSGALTSGVHTFPAVLQS | |
| | | SGLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 151 |
| | chain | QQKPGKAPKLLIYSASSLYSGVPSRFSGSRSGTDFTL | |
| | | TISSLQPEDFATYYCQQSSWSPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH22 | HCDR1 | FSSSYI | 153 |
| | HCDR2 | SIYSYSGYTS | 62 |
| | HCDR3 | YRFIGAM | 152 |
| | LCDR1 | SVSSA | 4 |
| | LCDR2 | SASSLYS | 5 |
| | LCDR3 | SHWSPI | 154 |
| | VH | EISEVQLVESGGGLVQPGGSLRLSCAASGFNFSSSYIH | 155 |
| | | WVRQAPGKGLEWVASIYSYSGYTSYADSVKGRFTIS | |
| | | ADTSKNTAYLQMNSLRAEDTAVYYCARYRFIGAMD | |
| | | YWGQGTLVTV | |
| | VL | SDIQMTQSPSSLSASVGDRVTITCRASQSVSSAVAWY | 156 |
| | | QQKPGKAPKLLIYSASSLYSGVPSRFSGSRSGTDFTL | |
| | | TISSLQPEDFATYYCQQSHWSPITFGQGTKVEIKRT | |
| | Full heavy | EISEVQLVESGGGLVQPGGSLRLSCAASGFNFSSSYIH | 157 |
| | chain | WVRQAPGKGLEWVASIYSYSGYTSYADSVKGRFTIS | |
| | | ADTSKNTAYLQMNSLRAEDTAVYYCARYRFIGAMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 158 |
| | chain | QQKPGKAPKLLIYSASSLYSGVPSRFSGSRSGTDFTL | |
| | | TISSLQPEDFATYYCQQSHWSPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH23 | HCDR1 | VSSSYI | 11 |
| | HCDR2 | SIYSYSGYTS | 62 |
| | HCDR3 | FWVWAM | 159 |
| | LCDR1 | SVSSA | 4 |
| | LCDR2 | SASSLYS | 5 |
| | LCDR3 | SSYIPL | 160 |
| | VH | EISEVQLVESGGGLVQPGGSLRLSCAASGFNVSSSYI | 161 |
| | | HWVRQAPGKGLEWVASIYSYSGYTSYADSVKGRFTI | |
| | | SADTSKNTAYLQMNSLRAEDTAVYYCARFWVWAM | |
| | | DYWGQGTLVTV | |
| | VL | SDIQMTQSPSSLSASVGDRVTITCRASQSVSSAVAWY | 162 |
| | | QQKPGKAPKLLIYSASSLYSGVPSRFSGSRSGTDFTL | |
| | | TISSLQPEDFATYYCQQSSYIPLTFGQGTKVEIKRT | |
| | Full heavy | EISEVQLVESGGGLVQPGGSLRLSCAASGFNVSSSYI | 163 |
| | chain | HWVRQAPGKGLEWVASIYSYSGYTSYADSVKGRFTI | |
| | | SADTSKNTAYLQMNSLRAEDTAVYYCARFWVWAM | |
| | | DYWGQGTLVTVSSASTKGPSVFPLAPSSKSTSGGTA | |
| | | ALGCLVKDYFPEPVTVSWNSGALTSGVHTFPAVLQS | |
| | | SGLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 164 |
| | chain | QQKPGKAPKLLIYSASSLYSGVPSRFSGSRSGTDFTL | |
| | | TISSLQPEDFATYYCQQSSYIPLTFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH24 | HCDR1 | VSSSSI | 21 |
| | HCDR2 | SIYSSYGYTS | 32 |
| | HCDR3 | GDRYVWAM | 165 |
| | LCDR1 | SVSSA | 4 |
| | LCDR2 | SASSLYS | 5 |
| | LCDR3 | SSSYPF | 166 |
| | VH | EISEVQLVESGGGLVQPGGSLRLSCAASGFNVSSSSIH | 167 |
| | | WVRQAPGKGLEWVASIYSSYGYTSYADSVKGRFTIS | |
| | | ADTSKNTAYLQMNSLRAEDTAVYYCARGDRYVWA | |
| | | MDYWGQGTLVTV | |
| | VL | SDIQMTQSPSSLSASVGDRVTITCRASQSVSSAVAWY | 168 |
| | | QQKPGKAPKLLIYSASSLYSGVPSRFSGSRSGTDFTL | |
| | | TISSLQPEDFATYYCQQSSSYPFTFGQGTKVEIKRT | |
| | Full heavy | EISEVQLVESGGGLVQPGGSLRLSCAASGFNVSSSSIH | 169 |
| | chain | WVRQAPGKGLEWVASIYSSYGYTSYADSVKGRFTIS | |
| | | ADTSKNTAYLQMNSLRAEDTAVYYCARGDRYVWA | |
| | | MDYWGQGTLVTVSSASTKGPSVFPLAPSSKSTSGGT | |
| | | AALGCLVKDYFPEPVTVSWNSGALTSGVHTFPAVLQ | |
| | | SSGLYSLSSVVTVPSSSLGTQTYICNVNHKPSNTKVD | |
| | | KKVEPKSCDKTHT | |
| | Full light | SDIQMTQSPSSLSASVGDRVTITCRASQSVSSAVAWY | 170 |
| | chain | QQKPGKAPKLLIYSASSLYSGVPSRFSGSRSGTDFTL | |
| | | TISSLQPEDFATYYCQQSSSYPFTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH25 | HCDR1 | FSYYSI | 172 |
| | HCDR2 | SISSYYGYTS | 171 |
| | HCDR3 | YFLGM | 173 |
| | LCDR1 | SVSSA | 4 |
| | LCDR2 | SASSLYS | 5 |
| | LCDR3 | YRYPPI | 174 |
| | VH | EISEVQLVESGGGLVQPGGSLRLSCAASGFNFSYYSI | 175 |
| | | HWVRQAPGKGLEWVASISSYYGYTSYADSVKGRFTI | |
| | | SADTSKNTAYLQMNSLRAEDTAVYYCARYFLGMD | |
| | | YWGQGTLVTV | |
| | VL | SDIQMTQSPSSLSASVGDRVTITCRASQSVSSAVAWY | 176 |
| | | QQKPGKAPKLLIYSASSLYSGVPSRFSGSRSGTDFTL | |
| | | TISSLQPEDFATYYCQQYRYPPITFGQGTKVEIKRT | |
| | Full heavy | EISEVQLVESGGGLVQPGGSLRLSCAASGFNFSYYSI | 177 |
| | chain | HWVRQAPGKGLEWVASISSYYGYTSYADSVKGRFTI | |
| | | SADTSKNTAYLQMNSLRAEDTAVYYCARYFLGMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 178 |
| | chain | QQKPGKAPKLLIYSASSLYSGVPSRFSGSRSGTDFTL | |
| | | TISSLQPEDFATYYCQQYRYPPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH26 | HCDR1 | SSSI | 179 |
| | HCDR2 | SISSSYSSTY | 180 |
| | HCDR3 | AWWWAL | 181 |
| | LCDR1 | SVSSA | 4 |
| | LCDR2 | SASSLYS | 5 |
| | LCDR3 | YVSSLI | 182 |
| | VH | EISEVQLVESGGGLVQPGGSLRLSCAASGFNSSSIHW | 183 |
| | | VRQAPGKGLEWVASISSSYSSTYYADSVKGRFTISA | |
| | | DTSKNTAYLQMNSLRAEDTAVYYCARAWWWALD | |
| | | YWGQGTLVTV | |
| | VL | SDIQMTQSPSSLSASVGDRVTITCRASQSVSSAVAWY | 184 |
| | | QQKPGKAPKLLIYSASSLYSGVPSRFSGSRSGTDFTL | |
| | | TISSLQPEDFATYYCQQYVSSLITFGQGTKVEIKRT | |
| | Full heavy | EISEVQLVESGGGLVQPGGSLRLSCAASGFNSSSIHW | 185 |
| | chain | VRQAPGKGLEWVASISSSYSSTYYADSVKGRFTISA | |
| | | DTSKNTAYLQMNSLRAEDTAVYYCARAWWWALD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHT | |
| | Full light | SDIQMTQSPSSLSASVGDRVTITCRASQSVSSAVAWY | 186 |
| | chain | QQKPGKAPKLLIYSASSLYSGVPSRFSGSRSGTDFTL | |
| | | TISSLQPEDFATYYCQQYVSSLITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH30 | HCDR1 | FSSSSI | 92 |
| | HCDR2 | SISPYYGYTS | 187 |
| | HCDR3 | TYYWKYGGPAM | 188 |
| | LCDR1 | SVSSA | 4 |
| | LCDR2 | SASSLYS | 5 |
| | LCDR3 | WSWYSLI | 189 |
| | VH | EISEVQLVESGGGLVQPGGSLRLSCAASGFNFSSSSI | 190 |
| | | HWVRQAPGKGLEWVASISPYYGYTSYADSVKGRF | |
| | | TISADTSKNTAYLQMNSLRAEDTAVYYCARTYYW | |
| | | KYGGPAMDYWGQGTLVTV | |
| | VL | SDIQMTQSPSSLSASVGDRVTITCRASQSVSSAVAWY | 191 |
| | | QQKPGKAPKLLIYSASSLYSGVPSRFSGSRSGTDFTL | |
| | | TISSLQPEDFATYYCQQWSWYSLITFGQGTKVEIKR | |
| | | T | |
| | Full heavy | EISEVQLVESGGGLVQPGGSLRLSCAASGFNFSSSSI | 192 |
| | chain | HWVRQAPGKGLEWVASISPYYGYTSYADSVKGRF | |
| | | TISADTSKNTAYLQMNSLRAEDTAVYYCARTYYW | |
| | | KYGGPAMDYWGQGTLVTVSSASTKGPSVFPLAPSS | |
| | | KSTSGGTAALGCLVKDYFPEPVTVSWNSGALTSGVH | |
| | | TFPAVLQSSGLYSLSSVVTVPSSSLGTQTYICNVNHK | |
| | | PSNTKVDKKVEPKSCDKTHT | |
| | Full light | SDIQMTQSPSSLSASVGDRVTITCRASQSVSSAVAWY | 193 |
| | chain | QQKPGKAPKLLIYSASSLYSGVPSRFSGSRSGTDFTL | |
| | | TISSLQPEDFATYYCQQWSWYSLITFGQGTKVEIKRT | |
| | | VAAPSVFIFPPSDSQLKSGTASVVCLLNNFYPREAKV | |
| | | QWKVDNALQSGNSQESVTEQDSKDSTYSLSSTLTLS | |
| | | KADYEKHKVYACEVTHQGLSSPVTKSFNRGEC | |
| GH32 | HCDR1 | FYSSYI | 127 |
| | HCDR2 | SIYSYSGYTS | 62 |
| | HCDR3 | YKYMGM | 194 |
| | LCDR1 | SVSSA | 4 |
| | LCDR2 | SASSLYS | 5 |
| | LCDR3 | SSWMPI | 195 |
| | VH | EISEVQLVESGGGLVQPGGSLRLSCAASGFNFYSSYI | 196 |
| | | HWVRQAPGKGLEWVASIYSYSGYTSYADSVKGRF | |
| | | TISADTSKNTAYLQMNSLRAEDTAVYYCARYKYM | |
| | | GMDYWGQGTLVTV | |
| | VL | SDIQMTQSPSSLSASVGDRVTITCRASQSVSSAVAWY | 197 |
| | | QQKPGKAPKLLIYSASSLYSGVPSRFSGSRSGTDFTL | |
| | | TISSLQPEDFATYYCQQSSWMPITFGQGTKVEIKRT | |
| | Full heavy | EISEVQLVESGGGLVQPGGSLRLSCAASGFNFYSSYI | 198 |
| | chain | HWVRQAPGKGLEWVASIYSYSGYTSYADSVKGRF | |
| | | TISADTSKNTAYLQMNSLRAEDTAVYYCARYKYM | |
| | | GMDYWGQGTLVTVSSASTKGPSVFPLAPSSKSTSGG | |
| | | TAALGCLVKDYFPEPVTVSWNSGALTSGVHTFPAVL | |
| | | QSSGLYSLSSVVTVPSSSLGTQTYICNVNHKPSNTKV | |
| | | DKKVEPKSCDKTHT | |
| | Full light | SDIQMTQSPSSLSASVGDRVTITCRASQSVSSAVAWY | 199 |
| | chain | QQKPGKAPKLLIYSASSLYSGVPSRFSGSRSGTDFTL | |
| | | TISSLQPEDFATYYCQQSSWMPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH33 | HCDR1 | FYSSYI | 127 |
| | HCDR2 | SIYSYSGYTS | 62 |
| | HCDR3 | YWYYYNGM | 200 |
| | LCDR1 | SVSSA | 4 |
| | LCDR2 | SASSLYS | 5 |
| | LCDR3 | IRYVPI | 20 |
| | VH | EISEVQLVESGGGLVQPGGSLRLSCAASGFNFYSSYI | 202 |
| | | HWVRQAPGKGLEWVASIYSYSGYTSYADSVKGRF | |
| | | TISADTSKNTAYLQMNSLRAEDTAVYYCARYWYY | |
| | | YNGMDYWGQGTLVTV | |
| | VL | SDIQMTQSPSSLSASVGDRVTITCRASQSVSSAVAWY | 203 |
| | | QQKPGKAPKLLIYSASSLYSGVPSRFSGSRSGTDFTL | |
| | | TISSLQPEDFATYYCQQIRYVPITFGQGTKVEIKRT | |
| | Full heavy | EISEVQLVESGGGLVQPGGSLRLSCAASGFNFYSSYI | 204 |
| | chain | HWVRQAPGKGLEWVASIYSYSGYTSYADSVKGRF | |
| | | TISADTSKNTAYLQMNSLRAEDTAVYYCARYWYY | |
| | | YNGMDYWGQGTLVTVSSASTKGPSVFPLAPSSKST | |
| | | SGGTAALGCLVKDYFPEPVTVSWNSGALTSGVHTFP | |
| | | AVLQSSGLYSLSSVVTVPSSSLGTQTYICNVNHKPSN | |
| | | TKVDKKVEPKSCDKTHT | |
| | Full light | SDIQMTQSPSSLSASVGDRVTITCRASQSVSSAVAWY | 205 |
| | chain | QQKPGKAPKLLIYSASSLYSGVPSRFSGSRSGTDFTL | |
| | | TISSLQPEDFATYYCQQIRYVPITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH34 | HCDR1 | FSYSSI | 206 |
| | HCDR2 | SIYPSYGYTS | 113 |
| | HCDR3 | YYYFGM | 207 |
| | LCDR1 | SVSSA | 4 |
| | LCDR2 | SASSLYS | 5 |
| | LCDR3 | SWSMPL | 208 |
| | VH | EISEVQLVESGGGLVQPGGSLRLSCAASGFNFSYSSI | 209 |
| | | HWVRQAPGKGLEWVASIYPSYGYTSYADSVKGRF | |
| | | TISADTSKNTAYLQMNSLRAEDTAVYYCARYYYFG | |
| | | MDYWGQGTLVTV | |
| | VL | SDIQMTQSPSSLSASVGDRVTITCRASQSVSSAVAWY | 210 |
| | | QQKPGKAPKLLIYSASSLYSGVPSRFSGSRSGTDFTL | |
| | | TISSLQPEDFATYYCQQSWSMPLTFGQGTKVEIKRT | |
| | Full heavy | EISEVQLVESGGGLVQPGGSLRLSCAASGFNFSYSSI | 211 |
| | chain | HWVRQAPGKGLEWVASIYPSYGYTSYADSVKGRF | |
| | | TISADTSKNTAYLQMNSLRAEDTAVYYCARYYYFG | |
| | | MDYWGQGTLVTVSSASTKGPSVFPLAPSSKSTSGGT | |
| | | AALGCLVKDYFPEPVTVSWNSGALTSGVHTFPAVLQ | |
| | | SSGLYSLSSVVTVPSSSLGTQTYICNVNHKPSNTKVD | |
| | | KKVEPKSCDKTHT | |
| | Full light | SDIQMTQSPSSLSASVGDRVTITCRASQSVSSAVAWY | 212 |
| | chain | QQKPGKAPKLLIYSASSLYSGVPSRFSGSRSGTDFTL | |
| | | TISSLQPEDFATYYCQQSWSMPLTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH35 | HCDR1 | VYYSSI | 213 |
| | HCDR2 | SIYSYSGYTS | 62 |
| | HCDR3 | WSYKAM | 214 |
| | LCDR1 | SVSSA | 4 |
| | LCDR2 | SASSLYS | 5 |
| | LCDR3 | DYYAPI | 215 |
| | VH | EISEVQLVESGGGLVQPGGSLRLSCAASGFNVYYSSI | 216 |
| | | HWVRQAPGKGLEWVASIYSYSGYTSYADSVKGRF | |
| | | TISADTSKNTAYLQMNSLRAEDTAVYYCARWSYK | |
| | | AMDYWGQGTLVTV | |
| | VL | SDIQMTQSPSSLSASVGDRVTITCRASQSVSSAVAWY | 217 |
| | | QQKPGKAPKLLIYSASSLYSGVPSRFSGSRSGTDFTL | |
| | | TISSLQPEDFATYYCQQDYYAPITFGQGTKVEIKRT | |
| | Full heavy | EISEVQLVESGGGLVQPGGSLRLSCAASGFNVYYSSI | 218 |
| | chain | HWVRQAPGKGLEWVASIYSYSGYTSYADSVKGRF | |
| | | TISADTSKNTAYLQMNSLRAEDTAVYYCARWSYK | |
| | | AMDYWGQGTLVTVSSASTKGPSVFPLAPSSKSTSGG | |
| | | TAALGCLVKDYFPEPVTVSWNSGALTSGVHTFPAVL | |
| | | QSSGLYSLSSVVTVPSSSLGTQTYICNVNHKPSNTKV | |
| | | DKKVEPKSCDKTHT | |
| | Full light | SDIQMTQSPSSLSASVGDRVTITCRASQSVSSAVAWY | 219 |
| | chain | QQKPGKAPKLLIYSASSLYSGVPSRFSGSRSGTDFTL | |
| | | TISSLQPEDFATYYCQQDYYAPITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH36 | HCDR1 | VYSYSI | 220 |
| | HCDR2 | SISSYSGYTS | 221 |
| | HCDR3 | GSSWWSYYVKAM | 222 |
| | LCDR1 | SVSSA | 4 |
| | LCDR2 | SASSLYS | 5 |
| | LCDR3 | GDYRKFLI | 223 |
| | VH | EISEVQLVESGGGLVQPGGSLRLSCAASGFNVYSYSI | 224 |
| | | HWVRQAPGKGLEWVASISSYSGYTSYADSVKGRF | |
| | | TISADTSKNTAYLQMNSLRAEDTAVYYCARGSSW | |
| | | WSYYVKAMDYWGQGTLVTV | |
| | VL | SDIQMTQSPSSLSASVGDRVTITCRASQSVSSAVAWY | 225 |
| | | QQKPGKAPKLLIYSASSLYSGVPSRFSGSRSGTDFTL | |
| | | TISSLQPEDFATYYCQQGDYRKFLITFGQGTKVEIKR | |
| | | T | |
| | Full heavy | EISEVQLVESGGGLVQPGGSLRLSCAASGFNVYSYSI | 226 |
| | chain | HWVRQAPGKGLEWVASISSYSGYTSYADSVKGRF | |
| | | TISADTSKNTAYLQMNSLRAEDTAVYYCARGSSW | |
| | | WSYYVKAMDYWGQGTLVTVSSASTKGPSVFPLAP | |
| | | SSKSTSGGTAALGCLVKDYFPEPVTVSWNSGALTSG | |
| | | VHTFPAVLQSSGLYSLSSVVTVPSSSLGTQTYICNVN | |
| | | HKPSNTKVDKKVEPKSCDKTHT | |
| | Full light | SDIQMTQSPSSLSASVGDRVTITCRASQSVSSAVAWY | 227 |
| | chain | QQKPGKAPKLLIYSASSLYSGVPSRFSGSRSGTDFTL | |
| | | TISSLQPEDFATYYCQQGDYRKFLITFGQGTKVEIKRT | |
| | | VAAPSVFIFPPSDSQLKSGTASVVCLLNNFYPREAKV | |
| | | QWKVDNALQSGNSQESVTEQDSKDSTYSLSSTLTLS | |
| | | KADYEKHKVYACEVTHQGLSSPVTKSFNRGEC | |
| GH37 | HCDR1 | FYYYYI | 228 |
| | HCDR2 | SISPYSGSTY | 229 |
| | HCDR3 | QEWHYSYMYGM | 230 |
| | LCDR1 | SVSSA | 4 |
| | LCDR2 | SASSLYS | 5 |
| | LCDR3 | GSFSLI | 231 |
| | VH | EISEVQLVESGGGLVQPGGSLRLSCAASGFNFYYYY | 232 |
| | | IHWVRQAPGKGLEWVASISPYSGSTYYADSVKGRF | |
| | | TISADTSKNTAYLQMNSLRAEDTAVYYCARQEWH | |
| | | YSYMYGMDYWGQGTLVTV | |
| | VL | SDIQMTQSPSSLSASVGDRVTITCRASQSVSSAVAWY | 233 |
| | | QQKPGKAPKLLIYSASSLYSGVPSRFSGSRSGTDFTL | |
| | | TISSLQPEDFATYYCQQGSFSLITFGQGTKVEIKRT | |
| | Full heavy | EISEVQLVESGGGLVQPGGSLRLSCAASGFNFYYYY | 234 |
| | chain | IHWVRQAPGKGLEWVASISPYSGSTYYADSVKGRF | |
| | | TISADTSKNTAYLQMNSLRAEDTAVYYCARQEWH | |
| | | YSYMYGMDYWGQGTLVTVSSASTKGPSVFPLAPSS | |
| | | KSTSGGTAALGCLVKDYFPEPVTVSWNSGALTSGVH | |
| | | TFPAVLQSSGLYSLSSVVTVPSSSLGTQTYICNVNHK | |
| | | PSNTKVDKKVEPKSCDKTHT | |
| | Full light | SDIQMTQSPSSLSASVGDRVTITCRASQSVSSAVAWY | 235 |
| | chain | QQKPGKAPKLLIYSASSLYSGVPSRFSGSRSGTDFTL | |
| | | TISSLQPEDFATYYCQQGSFSLITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |
| GH38 | HCDR1 | VYSSSI | 48 |
| | HCDR2 | SIYPSYGYTS | 113 |
| | HCDR3 | WHWGWGSYGWGM | 236 |
| | LCDR1 | SVSSA | 4 |
| | LCDR2 | SASSLYS | 5 |
| | LCDR3 | QWYVPI | 237 |
| | VH | EISEVQLVESGGGLVQPGGSLRLSCAASGFNVYSSSI | 238 |
| | | HWVRQAPGKGLEWVASIYPSYGYTSYADSVKGRF | |
| | | TISADTSKNTAYLQMNSLRAEDTAVYYCARWHWG | |
| | | WGSYGWGMDYWGQGTLVTV | |
| | VL | SDIQMTQSPSSLSASVGDRVTITCRASQSVSSAVAWY | 239 |
| | | QQKPGKAPKLLIYSASSLYSGVPSRFSGSRSGTDFTL | |
| | | TISSLQPEDFATYYCQQQWYVPITFGQGTKVEIKRT | |
| | Full heavy | EISEVQLVESGGGLVQPGGSLRLSCAASGFNVYSSSI | 240 |
| | chain | HWVRQAPGKGLEWVASIYPSYGYTSYADSVKGRF | |
| | | TISADTSKNTAYLQMNSLRAEDTAVYYCARWHWG | |
| | | WGSYGWGMDYWGQGTLVTVSSASTKGPSVFPLAP | |
| | | SSKSTSGGTAALGCLVKDYFPEPVTVSWNSGALTSG | |
| | | VHTFPAVLQSSGLYSLSSVVTVPSSSLGTQTYICNVN | |
| | | HKPSNTKVDKKVEPKSCDKTHT | |
| | Full light | SDIQMTQSPSSLSASVGDRVTITCRASQSVSSAVAWY | 241 |
| | chain | QQKPGKAPKLLIYSASSLYSGVPSRFSGSRSGTDFTL | |
| | | TISSLQPEDFATYYCQQQWYVPITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH40 | HCDR1 | FSYYSI | 172 |
| | HCDR2 | SISSYSGSTS | 242 |
| | HCDR3 | DEFTYWGWSYAF | 243 |
| | LCDR1 | SVSSA | 4 |
| | LCDR2 | SASSLYS | 5 |
| | LCDR3 | SEYGLI | 244 |
| | VH | EISEVQLVESGGGLVQPGGSLRLSCAASGFNFSYYSI | 245 |
| | | HWVRQAPGKGLEWVASISSYSGSTSYADSVKGRFT | |
| | | ISADTSKNTAYLQMNSLRAEDTAVYYCARDEFTYW | |
| | | GWSYAFDYWGQGTLVTV | |
| | VL | SDIQMTQSPSSLSASVGDRVTITCRASQSVSSAVAWY | 246 |
| | | QQKPGKAPKLLIYSASSLYSGVPSRFSGSRSGTDFTL | |
| | | TISSLQPEDFATYYCQQSEYGLITFGQGTKVEIKRT | |
| | Full heavy | EISEVQLVESGGGLVQPGGSLRLSCAASGFNFSYYSI | 247 |
| | chain | HWVRQAPGKGLEWVASISSYSGSTSYADSVKGRFT | |
| | | ISADTSKNTAYLQMNSLRAEDTAVYYCARDEFTYW | |
| | | GWSYAFDYWGQGTLVTVSSASTKGPSVFPLAPSSK | |
| | | STSGGTAALGCLVKDYFPEPVTVSWNSGALTSGVHT | |
| | | FPAVLQSSGLYSLSSVVTVPSSSLGTQTYICNVNHKP | |
| | | SNTKVDKKVEPKSCDKTHT | |
| | Full light | SDIQMTQSPSSLSASVGDRVTITCRASQSVSSAVAWY | 248 |
| | chain | QQKPGKAPKLLIYSASSLYSGVPSRFSGSRSGTDFTL | |
| | | TISSLQPEDFATYYCQQSEYGLITFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH41 | HCDR1 | FSSSSI | 92 |
| | HCDR2 | SISPYSGYTS | 249 |
| | HCDR3 | GYGGWYWWYTFAF | 250 |
| | LCDR1 | SVSSA | 4 |
| | LCDR2 | SASSLYS | 5 |
| | LCDR3 | SSSSELL | 251 |
| | VH | EISEVQLVESGGGLVQPGGSLRLSCAASGFNFSSSSI | 252 |
| | | HWVRQAPGKGLEWVASISPYSGYTSYADSVKGRF | |
| | | TISADTSKNTAYLQMNSLRAEDTAVYYCARGYGG | |
| | | WYWWYTFAFDYWGQGTLVTV | |
| | VL | SDIQMTQSPSSLSASVGDRVTITCRASQSVSSAVAWY | 253 |
| | | QQKPGKAPKLLIYSASSLYSGVPSRFSGSRSGTDFTL | |
| | | TISSLQPEDFATYYCQQSSSSELLTFGQGTKVEIKRT | |
| | Full heavy | EISEVQLVESGGGLVQPGGSLRLSCAASGFNFSSSSI | 254 |
| | chain | HWVRQAPGKGLEWVASISPYSGYTSYADSVKGRF | |
| | | TISADTSKNTAYLQMNSLRAEDTAVYYCARGYGG | |
| | | WYWWYTFAFDYWGQGTLVTVSSASTKGPSVFPLA | |
| | | PSSKSTSGGTAALGCLVKDYFPEPVTVSWNSGALTS | |
| | | GVHTFPAVLQSSGLYSLSSVVTVPSSSLGTQTYICNV | |
| | | NHKPSNTKVDKKVEPKSCDKTHT | |
| | Full light | SDIQMTQSPSSLSASVGDRVTITCRASQSVSSAVAWY | 255 |
| | chain | QQKPGKAPKLLIYSASSLYSGVPSRFSGSRSGTDFTL | |
| | | TISSLQPEDFATYYCQQSSSSELLTFGQGTKVEIKRTV | |
| | | AAPSVFIFPPSDSQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| GH42 | HCDR1 | FSSSYI | 153 |
| | HCDR2 | SISSSSGSTS | 78 |
| | HCDR3 | SPWYVYWYFGSAL | 256 |
| | LCDR1 | SVSSA | 4 |
| | LCDR2 | SASSLYS | 5 |
| | LCDR3 | SSSSLI | 106 |
| | VH | EISEVQLVESGGGLVQPGGSLRLSCAASGFNFSSSYI | 257 |
| | | HWVRQAPGKGLEWVASISSSSGSTSYADSVKGRFTI | |
| | | SADTSKNTAYLQMNSLRAEDTAVYYCARSPWYVY | |
| | | WYFGSALDYWGQGTLVTV | |
| | VL | SDIQMTQSPSSLSASVGDRVTITCRASQSVSSAVAWY | 108 |
| | | QQKPGKAPKLLIYSASSLYSGVPSRFSGSRSGTDFTL | |
| | | TISSLQPEDFATYYCQQSSSSLITFGQGTKVEIKRT | |
| | Full heavy | EISEVQLVESGGGLVQPGGSLRLSCAASGFNFSSSYI | 258 |
| | chain | HWVRQAPGKGLEWVASISSSSGSTSYADSVKGRFTI | |
| | | SADTSKNTAYLQMNSLRAEDTAVYYCARSPWYVY | |
| | | WYFGSALDYWGQGTLVTVSSASTKGPSVFPLAPSS | |
| | | KSTSGGTAALGCLVKDYFPEPVTVSWNSGALTSGVH | |
| | | TFPAVLQSSGLYSLSSVVTVPSSSLGTQTYICNVNHK | |
| | | PSNTKVDKKVEPKSCDKTHT | |
| | Full light | SDIQMTQSPSSLSASVGDRVTITCRASQSVSSAVAWY | 82 |
| | chain | QQKPGKAPKLLIYSASSLYSGVPSRFSGSRSGTDFTL | |
| | | TISSLQPEDFATYYCQQSSSSLITFGQGTKVEIKRTVA | |
| | | APSVFIFPPSDSQLKSGTASVVCLLNNFYPREAKVQW | |
| | | KVDNALQSGNSQESVTEQDSKDSTYSLSSTLTLSKA | |
| | | DYEKHKVYACEVTHQGLSSPVTKSFNRGEC | |

TABLE 2
Summary of SEQ ID NOs.

| | | | | | | | | | Heavy | Light |
|---|---|---|---|---|---|---|---|---|---|---|
| Clone | HCDR1 | HCDR2 | HCDR3 | LCDR1 | LCDR2 | LCDR3 | VH | VL | Chain | Chain |
| GH1 | 1 | 2 | 3 | 4 | 5 | 6 | 7 | 8 | 9 | 10 |
| GH2 | 11 | 12 | 13 | 4 | 5 | 14 | 15 | 16 | 17 | 18 |
| GH3 | 21 | 19 | 20 | 4 | 5 | 22 | 23 | 24 | 25 | 26 |
| GH4 | 11 | 32 | 27 | 4 | 5 | 28 | 29 | 30 | 31 | 33 |
| GH5 | 34 | 12 | 35 | 4 | 5 | 36 | 37 | 38 | 39 | 40 |
| GH6 | 41 | 32 | 42 | 4 | 5 | 43 | 44 | 45 | 46 | 47 |
| GH7 | 48 | 62 | 49 | 4 | 5 | 50 | 51 | 52 | 53 | 54 |
| GH8 | 71 | 55 | 56 | 4 | 5 | 57 | 58 | 59 | 60 | 61 |
| GH9 | 71 | 62 | 63 | 4 | 5 | 64 | 65 | 66 | 67 | 68 |
| GH10 | 91 | 69 | 70 | 4 | 5 | 72 | 73 | 74 | 75 | 76 |
| GH11 | 77 | 78 | 79 | 4 | 5 | 106 | 80 | 108 | 81 | 82 |
| GH12 | 83 | 84 | 85 | 4 | 5 | 86 | 87 | 88 | 89 | 90 |
| GH13 | 92 | 93 | 94 | 4 | 5 | 106 | 95 | 108 | 96 | 82 |
| GH14 | 91 | 12 | 97 | 4 | 5 | 98 | 99 | 100 | 101 | 102 |
| GH15 | 11 | 62 | 103 | 4 | 5 | 104 | 105 | 107 | 109 | 110 |
| GH16 | 111 | 113 | 112 | 4 | 5 | 114 | 115 | 116 | 117 | 118 |
| GH17 | 119 | 32 | 120 | 4 | 5 | 121 | 122 | 123 | 124 | 125 |
| GH18 | 127 | 62 | 126 | 4 | 5 | 128 | 129 | 130 | 131 | 132 |
| GH19 | 133 | 62 | 134 | 4 | 5 | 135 | 136 | 137 | 138 | 139 |
| GH20 | 11 | 62 | 140 | 4 | 5 | 141 | 142 | 143 | 144 | 145 |
| GH21 | 11 | 62 | 146 | 4 | 5 | 147 | 148 | 149 | 150 | 151 |
| GH22 | 153 | 62 | 152 | 4 | 5 | 154 | 155 | 156 | 157 | 158 |
| GH23 | 11 | 62 | 159 | 4 | 5 | 160 | 161 | 162 | 163 | 164 |
| GH24 | 21 | 32 | 165 | 4 | 5 | 166 | 167 | 168 | 169 | 170 |
| GH25 | 172 | 171 | 173 | 4 | 5 | 174 | 175 | 176 | 177 | 178 |
| GH26 | 179 | 180 | 181 | 4 | 5 | 182 | 183 | 184 | 185 | 186 |
| GH30 | 92 | 187 | 188 | 4 | 5 | 189 | 190 | 191 | 192 | 193 |
| GH32 | 127 | 62 | 194 | 4 | 5 | 195 | 196 | 197 | 198 | 199 |
| GH33 | 127 | 62 | 200 | 4 | 5 | 201 | 202 | 203 | 204 | 205 |
| GH34 | 206 | 113 | 207 | 4 | 5 | 208 | 209 | 210 | 211 | 212 |
| GH35 | 213 | 62 | 214 | 4 | 5 | 215 | 216 | 217 | 218 | 219 |
| GH36 | 220 | 221 | 222 | 4 | 5 | 223 | 224 | 225 | 226 | 227 |
| GH37 | 228 | 229 | 230 | 4 | 5 | 231 | 232 | 233 | 234 | 235 |
| GH38 | 48 | 113 | 236 | 4 | 5 | 237 | 238 | 239 | 240 | 241 |
| GH40 | 172 | 242 | 243 | 4 | 5 | 244 | 245 | 246 | 247 | 248 |
| GH41 | 92 | 249 | 250 | 4 | 5 | 251 | 252 | 253 | 254 | 255 |
| GH42 | 153 | 78 | 256 | 4 | 5 | 106 | 257 | 108 | 258 | 82 |

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### V. NUCLEIC ACIDS

In certain embodiments, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5x sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred embodiment, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VI. POLYPEPTIDE EXPRESSION

In some aspects, there are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### VII. CANCER THERAPY

In some aspects, the disclosed methods comprise administering a cancer therapy to a subject or patient. The cancer therapy may be chosen based on an expression level measurement, alone or in combination with the clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is aggressive cancer. In some aspects, the cancer is Stage I cancer. In some aspects, the cancer is Stage II cancer (e.g., IIA, IIB, IIC). In some aspects, the cancer is Stage III cancer (e.g., IIIA, IIIB, IIIC). In some aspects, the cancer is Stage IV cancer (e.g., IVA, IVB).

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

A. GH-targeting polypeptides

Aspects of the disclosure comprise administering one or more GH-targeting polypeptides of the present disclosure to a subject. A subject may be administered 1, 2, 3, 4, 5, 6, 7, 8, 9, 10, or more GH-targeting polypeptides of the disclosure. A GH-targeting polypeptide of the disclosure may be administered alone or in combination with any other cancer therapy known in the art and/or described herein. A GH-targeting polypeptide of the disclosure may be administered to a subject in various forms, including as a recombinant polypeptide, as a nucleic acid encoding the polypeptide, and as a cell (e.g., immune cell such as T cell or NK cell) expressing or capable of expressing the polypeptide.

### B. Combination Therapies

In some aspects, the methods may further comprise administration of an additional therapy such as radiotherapy, cancer immunotherapy, oncolytic virus, chemotherapies, hormone therapies, or surgery. Specific chemotherapies include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane), antimicrotubule agents, e.g., Paclitaxel (“Taxol”) and doxorubicin hydrochloride (“doxorubicin”), pyrimidine analogs, such as cytarabine (cytosine arabinoside), 5-fluorouracil (fluouracil; 5-FU) and floxuridine (fluorode-oxyuridine; FudR).

### VIII. ACROMEGALY THERAPY

In some aspects, the disclosed methods comprise administering an acromegaly therapy to a subject or patient. In some aspects, the acromegaly therapy is surgery (e.g., resection of pituitary adenomas). In some aspects, the acromegaly therapy is radiotherapy. In some aspects, the acromegaly therapy comprises treatment with a therapeutic agent. In some aspects, the therapeutic agent is a somatostatin analog (e.g., octreotide, lanreotide). In some aspects, the therapeutic agent is a dopamine receptor agonist (e.g., cabergoline). In some aspects, the therapeutic agent is a GHR antagonist (e.g., pegvisomant). In some aspects, the therapeutic agent is a GHR-targeting polypeptide of the disclosure (e.g., an antibody or antibody fragment that specifically binds to GHR). Any of these acromegaly therapies may also be excluded. Combinations of these therapies may also be administered.

### A. GH-Targeting Polypeptides

Aspects of the disclosure comprise administering one or more GH-targeting polypeptides (e.g., GH-targeting antibodies, GH-targeting antibody fragments, etc.) of the present disclosure to a subject. A subject may be administered 1, 2, 3, 4, 5, 6, 7, 8, 9, 10, or more GH-targeting polypeptides of the disclosure. A GH-targeting polypeptide of the disclosure may be administered alone or in combination with any other acromegaly therapy known in the art and/or described herein. A GH-targeting polypeptide of the disclosure may be administered to a subject in various forms, including as a recombinant polypeptide, as a nucleic acid encoding the polypeptide, and as a cell (e.g., immune cell such as T cell or NK cell) expressing or capable of expressing the polypeptide.

### IX. PHARMACEUTICAL COMPOSITIONS

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising GHR-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 M to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. DETECTABLE LABELS

In some aspects of this disclosure, it will be useful to detectably or therapeutically label a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### XI. SAMPLE PREPARATION

In certain aspects, methods involve obtaining or evaluating a sample from a subject. The sample may include a sample obtained from any source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XII. HOST CELLS

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### XIII. KITS

Certain aspects of the present invention also concern kits containing compositions of the disclosure or compositions to implement methods of the disclosure. In some embodiments, kits can be used to detect the presence of a cancer cell in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to GH, including polypeptides disclosed herein. For example, a kit may comprise at least 1, 2, 3, 4, 5, 6, 7, 8, 9, 10 or more antibodies or antibody fragments (e.g., Fabs) disclosed herein for detecting GH, in some cases for detecting GH on expressed by a cancer cell. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting GH, such as GH expressed by a cancer cell, in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XIV. EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

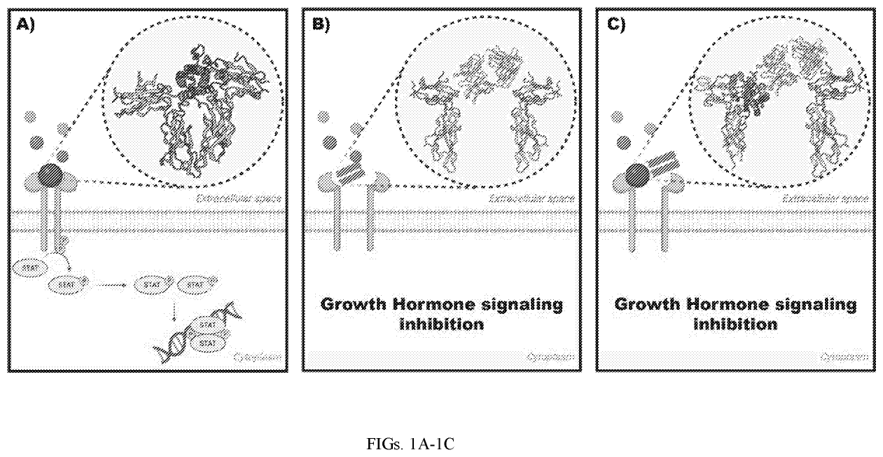

Acromegaly is a rare condition, where the production of Growth Hormone (GH) is not controlled, resulting in many physiological disorders. Certain aspects herein show the ability to inhibit the GH-mediated cellular signaling. GH binds to the single receptor through the “high affinity” site one, and then consequently to the second receptor by the “weaker affinity” site two, causing the receptor dimerization that induces intracellular signal transduction (FIG. 1A).

The inventors generated a cohort of antibodies that bind to the GHR monomer resulting in blocking GH binding site (FIG. 1B). These antibodies are characterized by an ultra-high affinity and a high inhibitory potency.

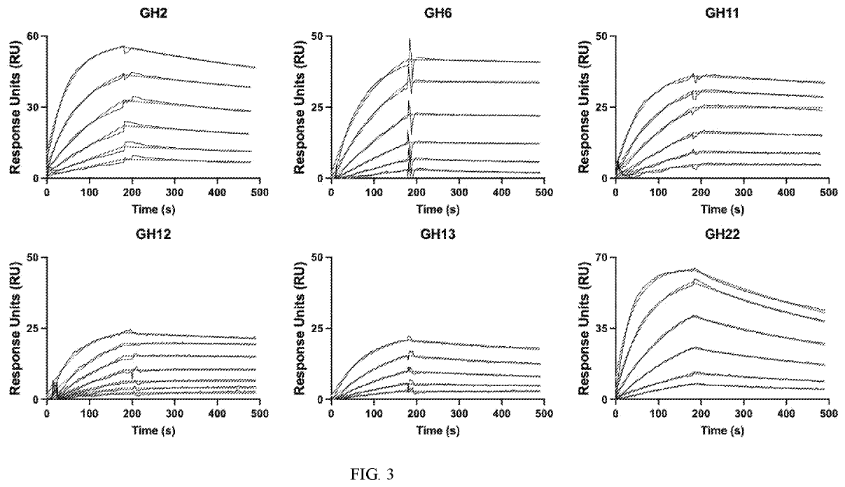

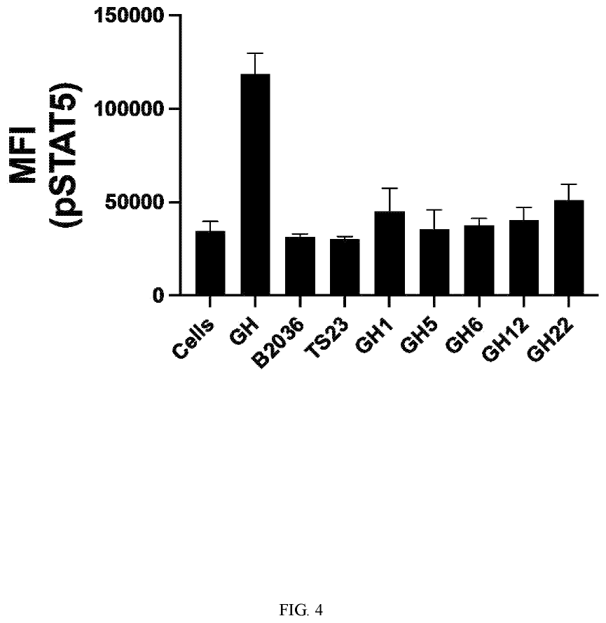

The inventors also generated antibodies that bind to the site 2 on the GH, and as a result allow formation of 1:1 complex with the receptor however do not grant the access for the second receptor binding and thus the dimerization (FIG. 1C). The engineered antibodies bind growth hormone with a very high affinity (FIG. 3) and inhibit the GH-mediated signaling in vitro (FIG. 4). Granting the access for 1:1 complex with the receptor is a bottom neck as binding to the free Growth Hormone would require constant dosing of the drug, however GHR-GH-Fab complex would potentially inhibit signaling for an extended period.

### Introduction and Methods

Phage display selection strategies were used with the human growth receptor as antigen to generate a diverse number of antibody inhibitors that competed with human growth hormone binding and reduced its downstream signaling. In vitro protein biochemistry, biophysical characterization, and cell-based assays were used to characterize and further develop the synthetic human antibodies. Most Fabs showed high-binding affinity, and a group of them showed inhibitory activity as determined by reduction of STAT5 phosphorylation in IM9 cells. These potent inhibitory antibodies and antibody fragments against GH may provide new strategies for therapeutic intervention in conditions like acromegalia, cancer, and longevity.

### Material and Methods

### Fabs and IgG Cloning

Briefly, CDR-containing regions were cloned into pSFV4 or pRH2.2 vectors, via Infusion cloning. Before converting FABs into IgG format, an N31D mutation was included to avoid glycosylation near CDR-H2 during IgG expression in human cells. For IgG cloning, the variable region from the light chain was per with forward primer 5-ttacgttcgtcgcggccgcagtcgcctccgatatccagatgacccagtc-3 (SEQ ID NO:499) and reverse primer 5-atggtgcagccaccgtacgtttgatctccaccttggtaccc-3 (SEQ ID NO:500). the per product was cloned into a psesta vector digested with noti and bsiwi enzymes, containing kappa light chain constant region. The heavy chain was per with forward primer 5-ttacgttcgtcgcggccgcagtcgccgagatctccgaggttcagctg-3 (SEQ ID NO:501) and reverse primer 5-tgggcccttggtgctagccgaggagacggtgaccagggttc-3 (SEQ ID NO:502). The PCR product from heavy chain was cloned into a pSCSTa vector using NotI and NheI enzymes; the heavy chain was cloned into a pSCSTa vector containing the Fc domain from human IgG1 scaffold. Cloning was done via In-Fusion cloning and positive cloning was verified by DNA sequencing. Fabs and IgG purification

Fabs were cloned in IPTG inducible vectors (pRH2.2 or pSFV4) for bacterial expression (E. coli BL21-Gold). Cells were grown for 24 hr at 30° C. in TB self-induction media (FisherScientific; BP9728-5) with added 0.01% glucose, 0.02% lactose, 1.25 mM MgSO₄, and 0.4% glycerol. Cells were harvested by centrifugation and sonicated in 50 mM Tris, pH=7.5, 200 mM NaCl, 2 mM PMSF. The centrifugation cleared lysates were heated at 65° C. for 30 min. Next, the lysate was centrifuged 30 min at 38,400 g to removed precipitated proteins. Lysates were filtered through a 0.2 μm filter and purified on AKTA purifier equipped with a Protein L resin (Cytiva; cat. no. 17547815). Next, peak fractions were loaded onto an ion exchange Resource S 1-ml column (GE Healthcare). After washing with 50 mM sodium acetate (pH 5.0), Fabs were eluted with a linear 0%-100% gradient of buffer containing 50 mM sodium acetate (pH 5.0) and 2 M sodium chloride. Fractions containing pure Fab were pooled, neutralized with 50 mM HEPES (pH 7.5), dialyzed against buffer containing 50 mM HEPES (pH 7.5) and 200 mM sodium chloride or 1×PBS, concentrated, and stored in aliquots at −80° C. For IgG expression and purification, Expi293 cells in 125 mL flask (Corning; cat no. 431143) at 2×10⁶ cells/mL in 10 mL were transfected with 7.5 μg each of light (kappa scaffold) and heavy chains (IgG1 scaffold) in pSCTa plasmid, using Opti-MEM media (Gibco; cat. no. 31985070) and FectoPro transfection reagent (Polyplus; cat. no 116-010), following the manufacturer recommendations. Next, 5 days after transfection the supernatant was collected, residual cells removed by centrifugation, and IgG purified by protein A resin (GE Healthcare; cat. no. 17127903) followed by glycine elution. Next, buffer was exchanged to 1×PBS and IgGs stored at 4° C. (months) or freezed at −80° C. for longer storage.

### Human Growth Hormone and Receptor Purification

Human growth hormone and B2036 variant were purified as described previously²⁴. Briefly, hormones in pET28 vector were transformed in E. coli BL21 (DE3) cells, grown in LB media and induced with IPTG (0.5 mM) for 16 hr at 16° C., cells were pelleted by centrifugation and lysed via sonication in buffer (50 mM Tris-HCl, pH 8.0, 0.5 mM EDTA, 0.1% Triton X-100). Next, lysate was purified with Ni-NTA agarose resin (Qiagen; cat. no. 30250), followed by size exclusion chromatography on a HiLoad 26/30 Superdex 200 column (GE Healthcare). Proteins were dialyzed and frozen at −80° C. Human growth hormone receptor and subdomain two (S2) constructs fused to Fc domain were expressed in Expi293 cells (ThermoFisher, A14527) by transient transfection using FectoPro transfection reagent (Polyplus; cat. no 116-010), cells were kept for 6 days before media harvested, then purified by protein A affinity chromatography using an AKTA purifier system. Protein was biotinylated in vitro using a commercial kit following the manufacturer recommendations (AVIDITY, Cat. No. BirA500).

### Phage Display Selection

The phage display selection was carried out as described before (Paduch, M., Methods 60, 3-14; Dominik, P. K., In Methods in Enzymology Membrane Proteins Engineering, Purification and Crystallization pp. 219-245). Briefly, human Growth Hormone was biotinylated using SNAP-tag as described previously (Slezak, T. Protein Sci 29, 141-156), and immobilized on streptavidin-coated paramagnetic beads (Promega; Z5482). In the first round, 500 nM of target was immobilized in 200 μL of beads and incubated with 1 mL of phage library (10×10¹¹ cfu) for 1 hr at room temperature with gentle shaking. The beads were washed three times with 0.5% BSA-PBST buffer, then used to infect E. coli XL-1 blue cells (Stratagene) in mid-log phase, for 20 min. Then media containing 100 μg/mL ampicillin and 10×⁹ p.f.u./mL of M13K07 helper phage (NEB) was added for overnight phage amplification at 37° C. For the next rounds, the phage was precipitated in 20% PEG/2.5M NaCl for 20 min on ice. Before each round, the precipitated phage was negatively selected against paramagnetic beads with bound hGH G120R mutant for 30 min with shaking to ensure the selected antibodies to bind to the “site 2” of GH. Target concentration for the next rounds were: 200 nM for second round, 50 nM third round, 10 nM fourth round, and 1 nM for the fifth round. During each round, phage was eluted with 0.1 M glycine, pH 2.6 and neutralized with Tris-HCl pH 8.5. Then phage was eluted and used for XL-1 infection and phage amplification overnight. Unique clones and best binder were screened via single point phage-ELISA.

### Phage and Protein ELISA

For phage ELISA, biotinylated target was immobilized at 50 nM in neutravidin coated plates (96 well plate, Geiner Bio, high binding). After blocking with 5% BSA, phage was added and incubated for 15 min, then well were extensively washed before incubation with anti-M13 antibody at 1:5,000 dilution in PBST. (Abcam; cat. No. b50370) or. For protein ELISA, FABs were added at different concentrations based on the experiment objectives (single vs multi point ELISA). The plates were washed and developed with TMB substrate (Thermo Scientific) and quenched with 1M HCL, followed by the absorbance measurement at 450 nm (FAB signal) and 570 nm (background subtraction). The protein ELISA protocol was conducted as described before.²⁸ For FAB detection, an anti-human FAB-HRP antibody (Jackson ImmunoResearch; Cat. No 209-035-097) was used at 1:500 dilution.

### SPR

Surface plasmon resonance (SPR) measurements were conducted in a MASS-1 (Bruker) instrument. The target GH-Fc (50 nM) with a 10× His-tag was captured in a Ni-NTA sensor chip. For affinity determination, FABs were run starting at 50 nM followed by 2-fold dilutions (8 dilutions). Flow rate was 30 ul/min and temperature 20° C. Sensograms were corrected through double referencing and fitting was done with a 1:1 binding model using the Sierra Analyzer (Bruker).

### Flow Cytometry

To measure the phosphorylation levels of endogenous STAT5 via flow cytometry, we followed published methods with some modifications.²⁹ IM9 cells were serum starved overnight (16 hr) in serum free media (RPMI, 2×L-glutamine, lx sodium pyruvate, lx non-essential amino acids, 1× penicillin/streptomycin). Next day cells were incubated with 200 nM FAB (50 uL per well) or with serial dilutions (IC50 determination). Next, cells were stimulated with hGH (35 nM) for 10 min at 37° C., 5% CO₂. Cells were fix with 4% PFA in 1×PBS for 10 min at R.T., washed with 1×PBS, permeabilized with 90% methanol for 30 min on ice, washed twice, then incubated with mixture (60:40 ratio) of STAT5-pY694 antibody (Cell Signaling; Cat. No. 9351S) and STAT5-pY694 (Cell Signaling; Cat. No. 9365S) at a 230:1 dilution (50 uL per well) overnight. Next day, cells were washed, incubated 30 min at 4° C. with secondary anti-rabbit antibody conjugated with Alexa-647 (Biolegend, poly 4064, cat. no. 406414), fix and read by flow cytometry (CytoFlex instrument; Beckman Coulter).

### Example 1—Fab Production

Fabs were expressed in the periplasm of E. coli BL21 cells for 4 hours at 37° C. post induction with 1 mM IPTG at OD₆₀₀=0.8-1. The cells were harvested by sonication. After centrifugation the supernatant was applied on the protein L affinity column (Cytiva). Proteins were eluted from the column with 0.1 M glycine, pH 2.6, and neutralized with 1M Tris-HCl, pH 8.5, and dialyzed overnight into PBS.

### Example 2—IgG Production

Full-length IgG were expressed in Expi293 cells at 2×10⁶ cells/mL. The heavy (IgG1 scaffold) and light chain (Kappa) were co-transfected using FectoPro transfection reagent (Polyplus) according to the manufacture's recommendation. Five days post transfection the supernatant was collected by centrifugation and IgG was purified using protein A resin (Cytiva Proteins were eluted from the column with 0.1 M glycine, pH 2.6, and neutralized with 1M Tris-HCl, pH 8.5, and dialyzed overnight into PBS.

### Example 3—GH Fabs Binding by ELISA Assay

The ELISA assay was performed to validate the GH Fabs binding to GH. 50 nM of biotinylated GH was immobilized on a 96-well neutravidin-coated plate (Geiner), followed by extensive blocking with BSA. Phage or purified Fabs were incubated on the plate for 15 minutes, washed three times, and incubated with either anti-M13 phage (Abcam) or anti-human Fab (Jackson Immunoresearch) antibody conjugated with HRP (in 1:5000 dilution in PBST) for 20 minutes in room temperature. The plates were then washed, developed with TMB substrate (Thermo Scientific), and quenched with 10% H3PO4, followed by the absorbance at A450 determination.

### Example 4—GH Fabs Binding by Surface Plasmon Resonance Analysis

All Surface plasmon resonance (SPR) analyses were performed on a MASS-1 (Bruker) to determine the association and desociation constants of GH Fabs. Target GH_Fc were immobilized via a 10× His-tag to a Ni-NTA sensor chip. Fabs in twofold dilutions were run as analytes at 30 μl/min flow rate at 20° C. Sensograms were corrected through double referencing and 1:1 binding model fit was done using Sierra Analyzer (Bruker).

### Example 5—GH Signaling Inhibition Assay

To determine the inhibitory activity of GH Fabs, the phosphorylation of endogenous STAT5 was measured by flow cytometry in IM9 cells. IM9 cells expressed GHR on the surface and were previously used to study GH-mediated receptor dimerization and signaling (Clayton et al., 1994). The day before the experiment, the IM9 cells were serum starved for 16 hours in RPMI media (supplemented with 2× L-glutamine, 1× sodium pyruvate, Ix non-essential amino acids, and Ix penicillin/streptomycin). To determine the GH Fabs inhibitory activity, the cells were incubated with 200 nM of GH Fabs for 30 minutes prior to the stimulation with 200 nM of GH for 10 minutes at 37° C. Next, cells were fixed and permeabilized with the 4% PFA and 90% methanol, extensively washed. The STAT5 phosphorylation was detected with the anti-STAT5-pY694 antibody (Cell Signaling) incubated overnight at 4° C. Next day, cells were washed, and the staining was measured by flow cytometry (CytoFlex instrument; Beckman Coulter) with the anti-rabbit secondary antibody conjugated with Alexa-647 (Biolegend). To determine the half maximum inhibitory concentration (IC50), the GH Fabs were incubated in a serial dilution while the staining protocol remained unchanged.

### Example 6—Kinetic Parameters of Binding Between Fab and Growth Hormone

| Fab | Kₒₙ (M⁻¹ s⁻¹) | K_{off} (s⁻¹) | K_{D} (nM) | χ² (RU²) |
|---|---|---|---|---|
| GH1 | 6.6 × 10⁵ | 9.7 × 10⁻⁴ | 1.5 | 0.5 |
| GH2 | 5.1 × 10⁵ | 4.6 × 10⁻⁴ | 0.9 | 0.9 |
| GH3 | 1.6 × 10⁶ | 3.3 × 10⁻² | 21 | 1.8 |
| GH4 | 4.1 × 10⁵ | 2.2 × 10⁻³ | 5.4 | 0.7 |
| GH5 | 4.4 × 10⁵ | 2.2 × 10⁻³ | 4.9 | 0.4 |
| GH6 | 6.4 × 10⁵ | 2.6 × 10⁻⁵ | 0.04 | 0.6 |
| GH7 | 2.9 × 10⁵ | 1.7 × 10⁻³ | 6.1 | 0.3 |
| GH9 | 1.6 × 10⁵ | 2.9 × 10⁻³ | 18 | 0.2 |
| GH10 | 8.3 × 10⁵ | 3.0 × 10⁻³ | 3.6 | 0.4 |
| GH11 | 4.3 × 10⁵ | 1.3 × 10⁻⁴ | 0.3 | 0.4 |
| GH12 | 4.9 × 10⁵ | 4.0 × 10⁻⁵ | 0.08 | 0.2 |
| GH13 | 1.2 × 10⁶ | 3.9 × 10⁻⁴ | 0.3 | 0.3 |
| GH14 | 4.7 × 10⁵ | 3.5 × 10⁻⁴ | 0.7 | 0.4 |
| GH15 | 2.0 × 10⁵ | 7.9 × 10⁻⁴ | 3.9 | 0.6 |
| GH16 | 4.0 × 10⁵ | 1.4 × 10⁻³ | 3.4 | 0.2 |
| GH18 | 2.9 × 10⁵ | 3.6 × 10⁻³ | 12.5 | 1.3 |
| GH19 | 4.5 × 10⁵ | 2.7 × 10⁻³ | 5.9 | 0.7 |
| GH20 | 5.0 × 10⁵ | 9.5 × 10⁻⁴ | 1.9 | 0.2 |
| GH21 | 2.7 × 10⁵ | 2.0 × 10⁻³ | 7.5 | 0.2 |
| GH22 | 1.2 × 10⁶ | 1.3 × 10⁻³ | 1.1 | 0.5 |
| GH23 | 2.8 × 10⁵ | 4.8 × 10⁻⁵ | 0.17 | 1.0 |
| GH24 | 2.4 × 10⁵ | 3.2 × 10⁻⁴ | 1.3 | 0.9 |
| GH25 | 1.7 × 10⁶ | 2.2 × 10⁻⁴ | 0.14 | 2.9 |
| GH32 | 6.1 × 10⁵ | 1.0 × 10⁻² | 17 | 1.0 |
| GH33 | 3.3 × 10⁵ | 1.9 × 10⁻³ | 5.8 | 0.4 |
| GH34 | 5.4 × 10⁵ | 1.1 × 10⁻² | 20 | 1.9 |
| GH35 | 4.9 × 10⁵ | 7.1 × 10⁻³ | 14 | 1.7 |
| GH38 | 1.9 × 10⁵ | 1.5 × 10⁻³ | 8.1 | 0.8 |
| GH41 | 9.0 × 10⁵ | 2.3 × 10⁻³ | 2.6 | 0.6 |
| GH42 | 1.1 × 10⁶ | 2.4 × 10⁻³ | 2.2 | 1.1 |

### Example 7—the Kinetic Parameters of the Binding Between Growth Hormone Site 1 and the Growth Hormone Receptor Influenced by Fab Binding to Growth Hormone Site 2

| Fab | Kₒₙ (M⁻¹ s⁻¹) | K_{off} (s⁻¹) | K_{D} (nM) | χ² (RU²) |
|---|---|---|---|---|
| None | 4.8 × 10⁵ | 7.0 × 10⁻⁴ | 1.46 | 2.2 |
| GH1 | 3.7 × 10⁵ | 5.6 × 10⁻⁵ | 0.15 | 0.1 |
| GH3 | 5.1 × 10⁵ | 5.7 × 10⁻³ | 0.12 | 0.1 |
| GH4 | 1.6 × 10⁵ | 1.6 × 10⁻⁴ | 0.95 | 0.1 |
| GH5 | 2.0 × 10⁵ | 3.6 × 10⁻⁵ | 0.18 | 0.1 |
| GH7 | 1.2 × 10⁵ | 2.2 × 10⁻⁶ | 0.02 | 0.2 |
| GH10 | 4.1 × 10⁵ | 1.4 × 10⁻⁴ | 0.33 | 0.3 |
| GH11 | 9.5 × 10⁴ | 4.9 × 10⁻⁵ | 0.52 | 0.2 |
| GH14 | 1.7 × 10⁵ | 7.3 × 10⁻⁵ | 0.44 | 0.2 |
| GH22 | 1.4 × 10⁵ | 4.1 × 10⁻⁴ | 3.0 | 0.1 |
| GH23 | 1.1 × 10⁵ | 1.8 × 10⁻⁵ | 0.15 | 0.2 |
| GH24 | 5.7 × 10⁴ | 2.4 × 10⁻⁵ | 0.42 | 0.1 |
| GH25 | 2.4 × 10⁵ | 4.6 × 10⁻⁵ | 0.19 | 0.5 |

The binding of selected Fabs to Site 2 on Growth Hormone alters the affinity between Growth Hormone Site 1 and the Growth Hormone Receptor.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and those cited elsewhere herein, to the extent that they provide exemplary procedural or other details supplementary to those set forth herein, are specifically incorporated herein by reference.
1. Dehkhoda, F., Lee, C. M. M., Medina, J., and Brooks, A. J. (2018). The Growth Hormone Receptor: Mechanism of Receptor Activation, Cell Signaling, and Physiological Aspects. Front. Endocrinol. 9:35.
2. Brooks, A. J., and Waters, M. J. (2010). The growth hormone receptor: mechanism of activation and clinical implications. Nat Rev Endocrinol 6, 515-525.
3. Wells, J. A. (1996). Binding in the growth hormone receptor complex. PNAS 93, 1-6.
4. Brown, R. J., Adams, J. J., Pelekanos, R. A., Wan, Y., McKinstry, W. J., Palethorpe, K., Seeber, R. M., Monks, T. A., Eidne, K. A., Parker, M. W., et al. (2005). Model for growth hormone receptor activation based on subunit rotation within a receptor dimer. Nature Structural & Molecular Biology 12, 814-821.
5. Herrington, J., Smit, L. S., Schwartz, J., and Carter-Su, C. (2000). The role of STAT proteins in growth hormone signaling. Oncogene 19, 2585-2597.
6. Guevara-Aguirre, J., Balasubramanian, P., Guevara-Aguirre, M., Wei, M., Madia, F., Cheng, C.-W., Hwang, D., Martin-Montalvo, A., Saavedra, J., Ingles, S., et al. (2011). Growth Hormone Receptor Deficiency Is Associated with a Major Reduction in Pro-Aging Signaling, Cancer, and Diabetes in Humans. Science Translational Medicine 3, 70ra13-70ra13.
7. Gebre-Medhin, M., Kindblom, L.-G., Wennbo, H., Törnell, J., and Meis-Kindblom, J. M. (2001). Growth Hormone Receptor Is Expressed in Human Breast Cancer. The American Journal of Pathology 158, 1217-1222.
8. Takahashi, Y., Kaji, H., Okimura, Y., Goji, K., Abe, H., and Chihara, K. (1996). Short Stature Caused by a Mutant Growth Hormone. New England Journal of Medicine 334, 432-436.
9. Trainer, P. J., Drake, W. M., Katznelson, L., Freda, P. U., Herman-Bonert, V., van der Lely, A. J., Dimaraki, E. V., Stewart, P. M., Friend, K. E., Vance, M. L., et al. (2009). Treatment of Acromegaly with the Growth Hormone-Receptor Antagonist Pegvisomant. http://dx.doi.org/10.1056/NEJM200004203421604. https://www.nejm.org/doi/10.1056/NEJM200004203421604?url_ver=Z39.88-2003&rfr_id=ori%3Arid%3Acrossref.org&rfr_dat=cr_pub%3Dwww.ncbi.nlm.nih.gov.
10. Colao, A., Ferone, D., Marzullo, P., and Lombardi, G. (2004). Systemic Complications of Acromegaly: Epidemiology, Pathogenesis, and Management. Endocr Rev 25, 102-152.
11. Melmed, S. (2009). Acromegaly pathogenesis and treatment. J Clin Invest 119, 3189-3202.
12. Colao, A., Auriemma, R. S., Lombardi, G., and Pivonello, R. (2011). Resistance to Somatostatin Analogs in Acromegaly. Endocrine Reviews 32, 247-271.
13. Murray, R. D., and Melmed, S. (2008). A critical analysis of clinically available somatostatin analog formulations for therapy of acromegaly. J Clin Endocrinol Metab 93, 2957-2968.
14. Burt, M. G., and Ho, K. K. Y. (2006). Newer options in the management of acromegaly. Internal Medicine Journal 36, 437-444.
15. Lowman, H. B., and Wells, J. A. (1993). Affinity Maturation of Human Growth Hormone by Monovalent Phage Display. Journal of Molecular Biology 234, 564-578.
16. Clark, R., Olson, K., Fuh, G., Marian, M., Mortensen, D., Teshima, G., Chang, S., Chu, H., Mukku, V., Canova-Davis, E., et al. (1996). Long-acting Growth Hormones Produced by Conjugation with Polyethylene Glycol. J. Biol. Chem. 271, 21969-21977.
17. Hodish, I., and Barkan, A. (2008). Long-term effects of pegvisomant in patients with acromegaly. Nat Rev Endocrinol 4, 324-332.
18. Moore, D. J., Adi, Y., Connock, M. J., and Bayliss, S. (2009). Clinical effectiveness and cost-effectiveness of pegvisomant for the treatment of acromegaly: a systematic review and economic evaluation. BMC Endocrine Disorders 9, 20.
19. BARNARD, R., BUNDESEN, P. G., RYLATT, D. B., and WATERS, M. J. (1984). Monoclonal Antibodies to the Rabbit Liver Growth Hormone Receptor: Production and Characterization*. Endocrinology 115, 1805-1813.
20. SIMPSON, J. S. A., HUGHES, J. P., and FRIESEN, H. G. (1983). A Monoclonal Antibody to the Growth Hormone Receptor of Rabbit Liver Membranes*. Endocrinology 112, 2137-2141.
21. Lan, H., Zheng, X., Khan, M. A., and Li, S. (2015). Anti-idiotypic antibody: A new strategy for the development of a growth hormone receptor antagonist. The International Journal of Biochemistry & Cell Biology 68, 101-108.
22. Fellouse, F. A., Esaki, K., Birtalan, S., Raptis, D., Cancasci, V. J., Koide, A., Jhurani, P., Vasser, M., Wiesmann, C., Kossiakoff, A. A., et al. (2007). High-throughput Generation of Synthetic Antibodies from Highly Functional Minimalist Phage-displayed Libraries. Journal of Molecular Biology 373, 924-940.
23. McCafferty, J., Griffiths, A. D., Winter, G., and Chiswell, D. J. (1990). Phage antibodies: filamentous phage displaying antibody variable domains. Nature 348, 552-554.
24. Kim, M.-J., Park, H. S., Seo, K. H., Yang, H.-J., Kim, S.-K., and Choi, J.-H. (2013). Complete Solubilization and Purification of Recombinant Human Growth Hormone Produced in Escherichia coli. PLOS ONE 8, e56168.
25. Mukherjee, S., Griffin, D. H., Horn, J. R., Rizk, S. S., Nocula-Lugowska, M., Malmqvist, M., Kim, S. S., and Kossiakoff, A. A. (2018). Engineered synthetic antibodies as probes to quantify the energetic contributions of ligand binding to conformational changes in proteins. J. Biol. Chem., jbc.RA117.000656.
26. Paduch, M., Koide, A., Uysal, S., Rizk, S. S., Koide, S., and Kossiakoff, A. A. (2013). Generating conformation-specific synthetic antibodies to trap proteins in selected functional states. Methods 60, 3-14.
27. Dominik, P. K., and Kossiakoff, A. A. (2015). Chapter Eleven—Phage Display Selections for Affinity Reagents to Membrane Proteins in Nanodiscs. In Methods in Enzymology Membrane Proteins-Engineering, Purification and Crystallization., A. K. Shukla, ed. (Academic Press), pp. 219-245.
28. Paduch, M., and Kossiakoff, A. A. (2017). Generating Conformation and Complex-Specific Synthetic Antibodies. Methods Mol Biol 1575, 93-119.
29. Malergue, F., van Agthoven, A., Scifo, C., Egan, D., and Strous, G. J. (2015). Automation of a Phospho-STAT5 Staining Procedure for Flow Cytometry for Application in Drug Discovery. J Biomol Screen 20, 416-421.
30. Clayton, P. E., Day, R. N., Silva, C. M., Hellmann, P., Day, K. H., and Thorner, M. O. (1994). Growth hormone induces tyrosine phosphorylation but does not alter insulin-like growth factor-I gene expression in human IM9 lymphocytes. J Mol Endocrinol 13, 127-136.
31. Fellouse, F. A., Esaki, K., Birtalan, S., Raptis, D., Cancasci, V. J., Koide, A., Jhurani, P., Vasser, M., Wiesmann, C., Kossiakoff, A. A., et al. (2007). High-throughput generation of synthetic antibodies from highly functional minimalist phage-displayed libraries. J Mol Biol 373, 924-940.
32. Miller, K. R., Koide, A., Leung, B., Fitzsimmons, J., Yoder, B., Yuan, H., Jay, M., Sidhu, S. S., Koide, S., and Collins, E. J. (2012). T cell receptor-like recognition of tumor in vivo by synthetic antibody fragment. PLoS One 7, e43746.
33. Paduch, M., Koide, A., Uysal, S., Rizk, S. S., Koide, S., and Kossiakoff, A. A. (2013). Generating conformation-specific synthetic antibodies to trap proteins in selected functional states. Methods 60, 3-14.
34. Schaefer, Z. P., Bailey, L. J., and Kossiakoff, A. A. (2016). A polar ring endows improved specificity to an antibody fragment. Protein Sci 25, 1290-1298.
35. Slezak, T., and Kossiakoff, A. A. (2021). Engineered Ultra-High Affinity Synthetic Antibodies for SARS-CoV-2 Neutralization and Detection. J Mol Biol 433, 166956.
36. Sidhu SS, Li B, Chen Y, Fellouse FA, Eigenbrot C, Fuh G. 2004. Phage-displayed antibody libraries of synthetic heavy chain complementarity determining regions. J Mol Biol. 338(2):299-310.
37. Kunkel TA. 1985. Rapid and efficient site-specific mutagenesis without phenotypic selection. Proc Natl Acad Sci USA. 82(2):488-492.

What is claimed is:

1. An antibody or antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region of a antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1.

2. The antibody or antigen binding fragment of claim 1, wherein the heavy chain variable region comprises a HCDR1, HCDR2, and HCDR3 having the amino acid sequence of an of a HCDR1, HCDR2, and HCDR3 of a clone of Table 1 and wherein the light chain variable region comprises a LCDR1, LCDR2, and LCDR3 comprising the amino acid sequence of the LCDR1, LCDR2, and LCDR3 from the light chain variable region of the same clone of Table 1.

3. The antibody or antigen binding fragment of claim 1 or 2, wherein the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

4. The antibody or antigen binding fragment of claim 1 or 2, wherein the HCDR1, HCDR2, HCDR2, LCDR1, LCDR2, and LCDR3 each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

5. The antibody or antigen binding fragment of any one of claims 1-4, wherein the heavy chain variable region comprises an amino acid sequence with at least 80% sequence identity to a heavy chain variable region of an antibody clone of Table 1 and/or the light chain variable region comprises an amino acid sequence with at least 80% sequence identity to the light chain variable region of the same antibody clone of Table 1.

6. The antibody or antigen binding fragment of claim 5, wherein the heavy chain variable region comprises the amino acid sequence of a heavy chain variable region of an antibody clone of Table 1 and/or the light chain variable region comprises the amino acid sequence of the same antibody clone of Table 1.

7. The antibody or antigen binding fragment of any one of claims 1-6, wherein the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone of Table 1, and the LFR1, LFR2, LFR3, and LFR4 comprises an amino acid sequence with at least 80% sequence identity to the LFR1, LFR2, LFR3, and LFR4, respectively, of the same antibody clone of Table 1.

8. The antibody or antigen binding fragment of any one of claims 1-6, wherein the HFR1, HFR2, HFR3, and HFR4 comprises the amino acid sequence of an HFR1, HFR2, HFR3, and HFR4, respectively, of an antibody clone of Table 1, and the LFR1, LFR2, LFR3, and LFR4 comprises the amino acid sequence of the LFR1, LFR2, LFR3, and LFR4, respectively, of the same antibody clone of Table 1.

9. The antibody or antigen binding fragment of any one of claims 1-8, wherein the antibody comprises a heavy chain and a light chain and wherein the heavy chain comprises an amino acid sequence with at least 70% sequence identity to a heavy chain of an antibody clone of Table 1 and the light chain comprises an amino acid sequence with at least 70% sequence identity to the light chain of the same antibody clone of Table 1.

10. The antibody or antigen binding fragment of claim 9, wherein the antibody comprises a heavy chain and a light chain and wherein the heavy chain comprises the amino acid sequence of an antibody clone of Table 1 and the light chain comprises the amino acid sequence of the same antibody clone of Table 1.

11. The antibody of any one of claims 1-10, wherein the antibody is human, chimeric, or humanized.

12. The antibody or antigen-binding fragment of any one of claims 1-11, wherein the antibody, or antigen binding fragment binds a Growth Hormone Receptor protein with a K_{D} of about 10⁻⁶ M/L to about 10⁻¹² M/L.

13. The antibody or antigen binding fragment of any one of claims 1-12, wherein the antibody is a neutralizing antibody.

14. The antibody or antigen binding fragment of any one of claims 1-13, wherein the antibody is a human antibody, humanized antibody, recombinant antibody, chimeric antibody, an antibody derivative, a veneered antibody, a diabody, a monoclonal antibody, a single domain antibody, or a single chain antibody.

15. The antigen binding fragment of any one of claims 1-13, wherein the antigen binding fragment is a single chain variable fragment (scFv), F(ab′)₂, Fab′, Fab, Fv, or rIgG.

16. A polypeptide comprising the antigen binding fragment of any one of claims 1-15.

17. The polypeptide of claim 16, wherein the polypeptide comprises at least two antigen binding fragments, wherein each antigen binding fragment is independently selected from an antigen binding fragment of any one of claims 1-15.

18. The polypeptide of claim 16 or 17, wherein the polypeptide is multivalent.

19. The polypeptide of any one of claims 16-18, wherein the polypeptide is bispecific.

20. A composition comprising the antibody or antigen binding fragment of any one of claims 1-15 or the polypeptide of any one of claims 16-19.

21. The composition of claim 20, wherein the composition comprises a pharmaceutical excipient.

22. The composition of claim 20 or 21, wherein the composition further comprises an adjuvant.

23. The composition of any one of claims 20-22, wherein the composition is formulated for parenteral, intravenous, subcutaneous, intramuscular, or intranasal administration.

24. The composition of any one of claims 1-23, wherein the composition comprises at least two antibodies or antigen binding fragments.

25. One or more nucleic acids encoding the antibody or antigen binding fragment of any one of claims 1-15 or the polypeptide of claim 19.

26. A vector comprising the nucleic acid(s) of claim 25.

27. A host cell comprising the nucleic acid of claim 25, or the vector of claim 26.

28. The host cell of claim 27, wherein the host cell is a human cell, B cell, T cell, Chinese hamster ovary, NS0 murine myeloma cell, or PER.C6 cell.

29. A method of a making a cell comprising transferring the nucleic acid(s) of claim 25 or the vector of claim 26 into a cell.

30. The method of claim 29, wherein the method further comprises culturing the cell under conditions that allow for expression of a polypeptide from the nucleic acid.

31. The method of claim 30, wherein the method further comprising isolating the expressed polypeptide.

32. The method of any one of claims 29-31, wherein the cell is a human cell, B cell, T cell, Chinese hamster ovary, NS0 murine myeloma cell, or PER.C6 cell.

33. A method for producing a polypeptide comprising transferring the nucleic acid(s) of claim 25 or the vector of claim 26 into a cell and isolating polypeptides expressed from the nucleic acid.

34. The method of claim 33, wherein the cell is a human cell, B cell, T cell, Chinese hamster ovary, NS0 murine myeloma cell, or PER.C6 cell.

35. A method for treating or preventing cancer in a subject, the method comprising administering to the subject the antibody or antigen binding fragment of any one of claims 1-15, the polypeptide of any one of claims 16-19, the composition of any one of claims 20-24, or the host cell of claim 27 or 28.

36. The method of claim 35, wherein the subject is a human subject.

37. The method of claim 35 or 36, wherein the subject has one or more symptoms of cancer.

38. The method of claim 35 or 36, wherein the subject does not have any symptoms of cancer.

39. The method of any one of claims 35-38, wherein the subject has been diagnosed with cancer.

40. The method of any one of claims 35-38, wherein the subject has not been diagnosed with cancer.

41. The method of any one of claims 35-40, wherein the subject has been previously treated for cancer.

42. The method of any one of claims 35-41, wherein the subject is administered an additional therapy.

43. The method of claim 42, wherein the additional therapy comprises radiotherapy, chemotherapy, or immunotherapy.

44. A method for treating or preventing acromegaly in a subject, the method comprising administering to the subject the antibody or antigen binding fragment of any one of claims 1-15, the polypeptide of any one of claims 16-19, the composition of any one of claims 20-24, or the host cell of claim 27 or 28.

45. The method of claim 44, wherein the subject is a human subject.

46. The method of claim 44 or 45, wherein the subject has one or more symptoms of acromegaly.

47. The method of claim 44 or 45, wherein the subject does not have one or more symptoms of acromegaly.

48. The method of claim 44 or 45, wherein the subject has been diagnosed with acromegaly.

49. The method of claim 44 or 45, wherein the subject has not been diagnosed with acromegaly.

50. The method of any of claims 44-49, wherein the subject has been previously treated for acromegaly.

51. The method of claim 50, wherein the subject was resistant to the previous treatment.

52. A method for evaluating a sample from a subject, the method comprising contacting a biological sample from the subject, or extract thereof, with at least one antibody, antigen binding fragment, or polypeptide of any one of claims 1-19.

53. The method of claim 52, wherein the at least one antibody, antigen binding fragment, or polypeptide is operatively linked to a detectable label.

54. The method of claim 52 or 53, wherein the method further comprises incubating the antibody, antigen binding fragment, or polypeptide under conditions that allow for the binding of the antibody, antigen binding fragment, or polypeptide to antigens in the biological sample or extract thereof.

55. The method of any one of claims 52-54, wherein the method further comprises detecting the binding of an antigen to the antibody, antigen binding fragment, or polypeptide.

56. The method of any one of claims 52-55, wherein the method further comprises contacting the biological sample with at least one capture antibody, antigen, or polypeptide.

57. The method of claim 56, wherein the at least one capture antibody, antigen binding fragment, or polypeptide comprises at least one antibody or antigen binding fragment of claims 1-15.

58. The method of claim 56 or 57, wherein the capture antibody or fragment is linked to a solid support.

59. The method of any one of claims 52-58, wherein the biological sample comprises a tissue sample or a blood sample.


SEQUENCE LISTING (258 sequences)

SEQ ID NO: 1 - protein, 6 aa, synthetic construct
FYYSSI

SEQ ID NO: 2 - protein, 10 aa, synthetic construct
YIYSYSGYTS

SEQ ID NO: 3 - protein, 7 aa, synthetic construct
YSWWGGM

SEQ ID NO: 4 - protein, 5 aa, synthetic construct
SVSSA

SEQ ID NO: 5 - protein, 7 aa, synthetic construct
SASSLYS

SEQ ID NO: 6 - protein, 6 aa, synthetic construct
SYSVPI

SEQ ID NO: 7 - protein, 119 aa, synthetic construct
EISEVQLVESGGGLVQPGGSLRLSCAASGFNFYYSSIHWVRQAPGKGLEWVAYIYSYSGYTSYADSVKGRFTISADTSKNTAYLQMNSLRAEDTAVYYCARYSWWGGMDYWGQGTLVTV

SEQ ID NO: 8 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYSVPITFGQGTKVEIKRT

SEQ ID NO: 9 - protein, 229 aa, synthetic construct
EISEVQLVESGGGLVQPGGSLRLSCAASGFNFYYSSIHWVRQAPGKGLEWVAYIYSYSGYTSYADSVKGRFTISADTSKNTAYLQMNSLRAEDTAVYYCARYSWWGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 10 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYSVPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 6 aa, synthetic construct
VSSSYI

SEQ ID NO: 12 - protein, 10 aa, synthetic construct
SIYSYYGYTS

SEQ ID NO: 13 - protein, 13 aa, synthetic construct
ENYGNWRSMYSGM

SEQ ID NO: 14 - protein, 6 aa, synthetic construct
SYFLPF

SEQ ID NO: 15 - protein, 125 aa, synthetic construct
EISEVQLVESGGGLVQPGGSLRLSCAASGFNVSSSYIHWVRQAPGKGLEWVASIYSYYGYTSYADSVKGRFTISADTSKNTAYLQMNSLRAEDTAVYYCARENYGNWRSMYSGMDYWGQGTLVTV

SEQ ID NO: 16 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYFLPFTFGQGTKVEIKRT

SEQ ID NO: 17 - protein, 235 aa, synthetic construct
EISEVQLVESGGGLVQPGGSLRLSCAASGFNVSSSYIHWVRQAPGKGLEWVASIYSYYGYTSYADSVKGRFTISADTSKNTAYLQMNSLRAEDTAVYYCARENYGNWRSMYSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 18 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYFLPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 10 aa, synthetic construct
YISSYYGYTS

SEQ ID NO: 20 - protein, 8 aa, synthetic construct
EGWYWWAM

SEQ ID NO: 21 - protein, 6 aa, synthetic construct
VSSSSI

SEQ ID NO: 22 - protein, 6 aa, synthetic construct
AWYLPI

SEQ ID NO: 23 - protein, 120 aa, synthetic construct
EISEVQLVESGGGLVQPGGSLRLSCAASGFNVSSSSIHWVRQAPGKGLEWVAYISSYYGYTSYADSVKGRFTISADTSKNTAYLQMNSLRAEDTAVYYCAREGWYWWAMDYWGQGTLVTV

SEQ ID NO: 24 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQAWYLPITFGQGTKVEIKRT

SEQ ID NO: 25 - protein, 230 aa, synthetic construct
EISEVQLVESGGGLVQPGGSLRLSCAASGFNVSSSSIHWVRQAPGKGLEWVAYISSYYGYTSYADSVKGRFTISADTSKNTAYLQMNSLRAEDTAVYYCAREGWYWWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 26 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQAWYLPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 12 aa, synthetic construct
ESWGSGYYTWAM

SEQ ID NO: 28 - protein, 6 aa, synthetic construct
SWRYPF

SEQ ID NO: 29 - protein, 124 aa, synthetic construct
EISEVQLVESGGGLVQPGGSLRLSCAASGFNVSSSYIHWVRQAPGKGLEWVASIYSSYGYTSYADSVKGRFTISADTSKNTAYLQMNSLRAEDTAVYYCARESWGSGYYTWAMDYWGQGTLVTV

SEQ ID NO: 30 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWRYPFTFGQGTKVEIKRT

SEQ ID NO: 31 - protein, 234 aa, synthetic construct
EISEVQLVESGGGLVQPGGSLRLSCAASGFNVSSSYIHWVRQAPGKGLEWVASIYSSYGYTSYADSVKGRFTISADTSKNTAYLQMNSLRAEDTAVYYCARESWGSGYYTWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 32 - protein, 10 aa, synthetic construct
SIYSSYGYTS

SEQ ID NO: 33 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWRYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 6 aa, synthetic construct
VSSYYI

SEQ ID NO: 35 - protein, 8 aa, synthetic construct
GDNYSWAM

SEQ ID NO: 36 - protein, 6 aa, synthetic construct
SWQYPF

SEQ ID NO: 37 - protein, 120 aa, synthetic construct
EISEVQLVESGGGLVQPGGSLRLSCAASGFNVSSYYIHWVRQAPGKGLEWVASIYSYYGYTSYADSVKGRFTISADTSKNTAYLQMNSLRAEDTAVYYCARGDNYSWAMDYWGQGTLVTV

SEQ ID NO: 38 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWQYPFTFGQGTKVEIKRT

SEQ ID NO: 39 - protein, 230 aa, synthetic construct
EISEVQLVESGGGLVQPGGSLRLSCAASGFNVSSYYIHWVRQAPGKGLEWVASIYSYYGYTSYADSVKGRFTISADTSKNTAYLQMNSLRAEDTAVYYCARGDNYSWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWQYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 6 aa, synthetic construct
VSYSSI

SEQ ID NO: 42 - protein, 8 aa, synthetic construct
GHGSMWAM

SEQ ID NO: 43 - protein, 6 aa, synthetic construct
SWSLPF

SEQ ID NO: 44 - protein, 120 aa, synthetic construct
EISEVQLVESGGGLVQPGGSLRLSCAASGFNVSYSSIHWVRQAPGKGLEWVASIYSSYGYTSYADSVKGRFTISADTSKNTAYLQMNSLRAEDTAVYYCARGHGSMWAMDYWGQGTLVTV

SEQ ID NO: 45 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSLPFTFGQGTKVEIKRT

SEQ ID NO: 46 - protein, 230 aa, synthetic construct
EISEVQLVESGGGLVQPGGSLRLSCAASGFNVSYSSIHWVRQAPGKGLEWVASIYSSYGYTSYADSVKGRFTISADTSKNTAYLQMNSLRAEDTAVYYCARGHGSMWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 47 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSLPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 6 aa, synthetic construct
VYSSSI

SEQ ID NO: 49 - protein, 8 aa, synthetic construct
GRYSWFAL

SEQ ID NO: 50 - protein, 6 aa, synthetic construct
YWSFPI

SEQ ID NO: 51 - protein, 120 aa, synthetic construct
EISEVQLVESGGGLVQPGGSLRLSCAASGFNVYSSSIHWVRQAPGKGLEWVASIYSYSGYTSYADSVKGRFTISADTSKNTAYLQMNSLRAEDTAVYYCARGRYSWFALDYWGQGTLVTV

SEQ ID NO: 52 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYWSFPITFGQGTKVEIKRT

SEQ ID NO: 53 - protein, 230 aa, synthetic construct
EISEVQLVESGGGLVQPGGSLRLSCAASGFNVYSSSIHWVRQAPGKGLEWVASIYSYSGYTSYADSVKGRFTISADTSKNTAYLQMNSLRAEDTAVYYCARGRYSWFALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 54 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYWSFPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 10 aa, synthetic construct
SISSSYGYTS

SEQ ID NO: 56 - protein, 8 aa, synthetic construct
GYYYSYAM

SEQ ID NO: 57 - protein, 6 aa, synthetic construct
SWSQPF

SEQ ID NO: 58 - protein, 120 aa, synthetic construct
EISEVQLVESGGGLVQPGGSLRLSCAASGFNVYSSYIHWVRQAPGKGLEWVASISSSYGYTSYADSVKGRFTISADTSKNTAYLQMNSLRAEDTAVYYCARGYYYSYAMDYWGQGTLVTV

SEQ ID NO: 59 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSQPFTFGQGTKVEIKRT

SEQ ID NO: 60 - protein, 230 aa, synthetic construct
EISEVQLVESGGGLVQPGGSLRLSCAASGFNVYSSYIHWVRQAPGKGLEWVASISSSYGYTSYADSVKGRFTISADTSKNTAYLQMNSLRAEDTAVYYCARGYYYSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 61 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSQPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 10 aa, synthetic construct
SIYSYSGYTS

SEQ ID NO: 63 - protein, 6 aa, synthetic construct
HSSWAM

SEQ ID NO: 64 - protein, 6 aa, synthetic construct
SSFVPL

SEQ ID NO: 65 - protein, 118 aa, synthetic construct
EISEVQLVESGGGLVQPGGSLRLSCAASGFNVYSSYIHWVRQAPGKGLEWVASIYSYSGYTSYADSVKGRFTISADTSKNTAYLQMNSLRAEDTAVYYCARHSSWAMDYWGQGTLVTV

SEQ ID NO: 66 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSFVPLTFGQGTKVEIKRT

SEQ ID NO: 67 - protein, 228 aa, synthetic construct
EISEVQLVESGGGLVQPGGSLRLSCAASGFNVYSSYIHWVRQAPGKGLEWVASIYSYSGYTSYADSVKGRFTISADTSKNTAYLQMNSLRAEDTAVYYCARHSSWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 68 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSFVPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 10 aa, synthetic construct
YISPYSGYTS

SEQ ID NO: 70 - protein, 13 aa, synthetic construct
IAYKWYMPYYYGM

SEQ ID NO: 71 - protein, 6 aa, synthetic construct
VYSSYI

SEQ ID NO: 72 - protein, 6 aa, synthetic construct
SLSSLV

SEQ ID NO: 73 - protein, 125 aa, synthetic construct
EISEVQLVESGGGLVQPGGSLRLSCAASGFNFYSSSIHWVRQAPGKGLEWVAYISPYSGYTSYADSVKGRFTISADTSKNTAYLQMNSLRAEDTAVYYCARIAYKWYMPYYYGMDYWGQGTLVTV

SEQ ID NO: 74 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSLSSLVTFGQGTKVEIKRT

SEQ ID NO: 75 - protein, 235 aa, synthetic construct
EISEVQLVESGGGLVQPGGSLRLSCAASGFNFYSSSIHWVRQAPGKGLEWVAYISPYSGYTSYADSVKGRFTISADTSKNTAYLQMNSLRAEDTAVYYCARIAYKWYMPYYYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 76 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSLSSLVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 6 aa, synthetic construct
IYSSSI

SEQ ID NO: 78 - protein, 10 aa, synthetic construct
SISSSSGSTS

SEQ ID NO: 79 - protein, 14 aa, synthetic construct
KYSGYYSNWYYPAL

SEQ ID NO: 80 - protein, 126 aa, synthetic construct
EISEVQLVESGGGLVQPGGSLRLSCAASGFNIYSSSIHWVRQAPGKGLEWVASISSSSGSTSYADSVKGRFTISADTSKNTAYLQMNSLRAEDTAVYYCARKYSGYYSNWYYPALDYWGQGTLVTV

SEQ ID NO: 81 - protein, 236 aa, synthetic construct
EISEVQLVESGGGLVQPGGSLRLSCAASGFNIYSSSIHWVRQAPGKGLEWVASISSSSGSTSYADSVKGRFTISADTSKNTAYLQMNSLRAEDTAVYYCARKYSGYYSNWYYPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 82 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 6 aa, synthetic construct
LSSSYI

SEQ ID NO: 84 - protein, 10 aa, synthetic construct
SIYPYSGYTS

SEQ ID NO: 85 - protein, 7 aa, synthetic construct
MYWTGAL

SEQ ID NO: 86 - protein, 6 aa, synthetic construct
SSYSPI

SEQ ID NO: 87 - protein, 119 aa, synthetic construct
EISEVQLVESGGGLVQPGGSLRLSCAASGFNLSSSYIHWVRQAPGKGLEWVASIYPYSGYTSYADSVKGRFTISADTSKNTAYLQMNSLRAEDTAVYYCARMYWTGALDYWGQGTLVTV

SEQ ID NO: 88 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSYSPITFGQGTKVEIKRT

SEQ ID NO: 89 - protein, 229 aa, synthetic construct
EISEVQLVESGGGLVQPGGSLRLSCAASGFNLSSSYIHWVRQAPGKGLEWVASIYPYSGYTSYADSVKGRFTISADTSKNTAYLQMNSLRAEDTAVYYCARMYWTGALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 90 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSYSPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 6 aa, synthetic construct
FYSSSI

SEQ ID NO: 92 - protein, 6 aa, synthetic construct
FSSSSI

SEQ ID NO: 93 - protein, 10 aa, synthetic construct
SISSYYSSTY

SEQ ID NO: 94 - protein, 12 aa, synthetic construct
QYTSPWHWYKAM

SEQ ID NO: 95 - protein, 124 aa, synthetic construct
EISEVQLVESGGGLVQPGGSLRLSCAASGFNFSSSSIHWVRQAPGKGLEWVASISSYYSSTYYADSVKGRFTISADTSKNTAYLQMNSLRAEDTAVYYCARQYTSPWHWYKAMDYWGQGTLVTV

SEQ ID NO: 96 - protein, 234 aa, synthetic construct
EISEVQLVESGGGLVQPGGSLRLSCAASGFNFSSSSIHWVRQAPGKGLEWVASISSYYSSTYYADSVKGRFTISADTSKNTAYLQMNSLRAEDTAVYYCARQYTSPWHWYKAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 7 aa, synthetic construct
SGGYIGF

SEQ ID NO: 98 - protein, 6 aa, synthetic construct
YWEYPF

SEQ ID NO: 99 - protein, 119 aa, synthetic construct
EISEVQLVESGGGLVQPGGSLRLSCAASGFNFYSSSIHWVRQAPGKGLEWVASIYSYYGYTSYADSVKGRFTISADTSKNTAYLQMNSLRAEDTAVYYCARSGGYIGFDYWGQGTLVTV

SEQ ID NO: 100 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYWEYPFTFGQGTKVEIKRT

SEQ ID NO: 101 - protein, 229 aa, synthetic construct
EISEVQLVESGGGLVQPGGSLRLSCAASGFNFYSSSIHWVRQAPGKGLEWVASIYSYYGYTSYADSVKGRFTISADTSKNTAYLQMNSLRAEDTAVYYCARSGGYIGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYWEYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 8 aa, synthetic construct
TSYGRGAL

SEQ ID NO: 104 - protein, 6 aa, synthetic construct
WSWYPI

SEQ ID NO: 105 - protein, 120 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARTSYGRGALDYWGQGTLVTV

SEQ ID NO: 106 - protein, 6 aa, synthetic construct
SSSSLI

SEQ ID NO: 107 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQWSWYPITFGQGTKVEIKRT

SEQ ID NO: 108 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRT

SEQ ID NO: 109 - protein, 230 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARTSYGRGALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 110 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQWSWYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 6 aa, synthetic construct
LSSYYI

SEQ ID NO: 112 - protein, 12 aa, synthetic construct
WGKWYGYPFSGL

SEQ ID NO: 113 - protein, 10 aa, synthetic construct
SIYPSYGYTS

SEQ ID NO: 114 - protein, 6 aa, synthetic construct
DWYFPI

SEQ ID NO: 115 - protein, 124 aa, synthetic construct
EISEVQLVESGGGLVQPGGSLRLSCAASGFNLSSYYIHWVRQAPGKGLEWVASIYPSYGYTSYADSVKGRFTISADTSKNTAYLQMNSLRAEDTAVYYCARWGKWYGYPFSGLDYWGQGTLVTV

SEQ ID NO: 116 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQDWYFPITFGQGTKVEIKRT

SEQ ID NO: 117 - protein, 234 aa, synthetic construct
EISEVQLVESGGGLVQPGGSLRLSCAASGFNLSSYYIHWVRQAPGKGLEWVASIYPSYGYTSYADSVKGRFTISADTSKNTAYLQMNSLRAEDTAVYYCARWGKWYGYPFSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 118 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQDWYFPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 4 aa, synthetic construct
ISSI

SEQ ID NO: 120 - protein, 6 aa, synthetic construct
WLYEAM

SEQ ID NO: 121 - protein, 6 aa, synthetic construct
FWFFPI

SEQ ID NO: 122 - protein, 116 aa, synthetic construct
EISEVQLVESGGGLVQPGGSLRLSCAASGFNISSIHWVRQAPGKGLEWVASIYSSYGYTSYADSVKGRFTISADTSKNTAYLQMNSLRAEDTAVYYCARWLYEAMDYWGQGTLVTV

SEQ ID NO: 123 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQFWFFPITFGQGTKVEIKRT

SEQ ID NO: 124 - protein, 226 aa, synthetic construct
EISEVQLVESGGGLVQPGGSLRLSCAASGFNISSIHWVRQAPGKGLEWVASIYSSYGYTSYADSVKGRFTISADTSKNTAYLQMNSLRAEDTAVYYCARWLYEAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 125 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQFWFFPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 7 aa, synthetic construct
YKEPFGM

SEQ ID NO: 127 - protein, 6 aa, synthetic construct
FYSSYI

SEQ ID NO: 128 - protein, 6 aa, synthetic construct
SNYMPL

SEQ ID NO: 129 - protein, 119 aa, synthetic construct
EISEVQLVESGGGLVQPGGSLRLSCAASGFNFYSSYIHWVRQAPGKGLEWVASIYSYSGYTSYADSVKGRFTISADTSKNTAYLQMNSLRAEDTAVYYCARYKEPFGMDYWGQGTLVTV

SEQ ID NO: 130 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSNYMPLTFGQGTKVEIKRT

SEQ ID NO: 131 - protein, 229 aa, synthetic construct
EISEVQLVESGGGLVQPGGSLRLSCAASGFNFYSSYIHWVRQAPGKGLEWVASIYSYSGYTSYADSVKGRFTISADTSKNTAYLQMNSLRAEDTAVYYCARYKEPFGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 132 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSNYMPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 6 aa, synthetic construct
FSYSYI

SEQ ID NO: 134 - protein, 6 aa, synthetic construct
YKWWAM

SEQ ID NO: 135 - protein, 6 aa, synthetic construct
SGSWPI

SEQ ID NO: 136 - protein, 118 aa, synthetic construct
EISEVQLVESGGGLVQPGGSLRLSCAASGFNFSYSYIHWVRQAPGKGLEWVASIYSYSGYTSYADSVKGRFTISADTSKNTAYLQMNSLRAEDTAVYYCARYKWWAMDYWGQGTLVTV

SEQ ID NO: 137 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSGSWPITFGQGTKVEIKRT

SEQ ID NO: 138 - protein, 228 aa, synthetic construct
EISEVQLVESGGGLVQPGGSLRLSCAASGFNFSYSYIHWVRQAPGKGLEWVASIYSYSGYTSYADSVKGRFTISADTSKNTAYLQMNSLRAEDTAVYYCARYKWWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 139 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSGSWPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 6 aa, synthetic construct
YKWYAM

SEQ ID NO: 141 - protein, 6 aa, synthetic construct
SSWYPI

SEQ ID NO: 142 - protein, 118 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARYKWYAMDYWGQGTLVTV

SEQ ID NO: 143 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWYPITFGQGTKVEIKRT

SEQ ID NO: 144 - protein, 228 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARYKWYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 145 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 6 aa, synthetic construct
YMYYAM

SEQ ID NO: 147 - protein, 6 aa, synthetic construct
SSWSPI

SEQ ID NO: 148 - protein, 118 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARYMYYAMDYWGQGTLVTV

SEQ ID NO: 149 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWSPITFGQGTKVEIKRT

SEQ ID NO: 150 - protein, 228 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARYMYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 151 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWSPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 7 aa, synthetic construct
YRFIGAM

SEQ ID NO: 153 - protein, 6 aa, synthetic construct
FSSSYI

SEQ ID NO: 154 - protein, 6 aa, synthetic construct
SHWSPI

SEQ ID NO: 155 - protein, 119 aa, synthetic construct
EISEVQLVESGGGLVQPGGSLRLSCAASGFNFSSSYIHWVRQAPGKGLEWVASIYSYSGYTSYADSVKGRFTISADTSKNTAYLQMNSLRAEDTAVYYCARYRFIGAMDYWGQGTLVTV

SEQ ID NO: 156 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSHWSPITFGQGTKVEIKRT

SEQ ID NO: 157 - protein, 229 aa, synthetic construct
EISEVQLVESGGGLVQPGGSLRLSCAASGFNFSSSYIHWVRQAPGKGLEWVASIYSYSGYTSYADSVKGRFTISADTSKNTAYLQMNSLRAEDTAVYYCARYRFIGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 158 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSHWSPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 6 aa, synthetic construct
FWVWAM

SEQ ID NO: 160 - protein, 6 aa, synthetic construct
SSYIPL

SEQ ID NO: 161 - protein, 118 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARFWVWAMDYWGQGTLVTV

SEQ ID NO: 162 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSYIPLTFGQGTKVEIKRT

SEQ ID NO: 163 - protein, 228 aa, synthetic construct
EISEVQLVESGGGLVQPGGSLRLSCAASGFNVSSSYIHWVRQAPGKGLEWVASIYSYSGYTSYADSVKGRFTISADTSKNTAYLQMNSLRAEDTAVYYCARFWVWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 164 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSYIPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 8 aa, synthetic construct
GDRYVWAM

SEQ ID NO: 166 - protein, 6 aa, synthetic construct
SSSYPF

SEQ ID NO: 167 - protein, 120 aa, synthetic construct
EISEVQLVESGGGLVQPGGSLRLSCAASGFNVSSSSIHWVRQAPGKGLEWVASIYSSYGYTSYADSVKGRFTISADTSKNTAYLQMNSLRAEDTAVYYCARGDRYVWAMDYWGQGTLVTV

SEQ ID NO: 168 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSSYPFTFGQGTKVEIKRT

SEQ ID NO: 169 - protein, 230 aa, synthetic construct
EISEVQLVESGGGLVQPGGSLRLSCAASGFNVSSSSIHWVRQAPGKGLEWVASIYSSYGYTSYADSVKGRFTISADTSKNTAYLQMNSLRAEDTAVYYCARGDRYVWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 170 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSSYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 10 aa, synthetic construct
SISSYYGYTS

SEQ ID NO: 172 - protein, 6 aa, synthetic construct
FSYYSI

SEQ ID NO: 173 - protein, 5 aa, synthetic construct
YFLGM

SEQ ID NO: 174 - protein, 6 aa, synthetic construct
YRYPPI

SEQ ID NO: 175 - protein, 117 aa, synthetic construct
EISEVQLVESGGGLVQPGGSLRLSCAASGFNFSYYSIHWVRQAPGKGLEWVASISSYYGYTSYADSVKGRFTISADTSKNTAYLQMNSLRAEDTAVYYCARYFLGMDYWGQGTLVTV

SEQ ID NO: 176 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYRYPPITFGQGTKVEIKRT

SEQ ID NO: 177 - protein, 227 aa, synthetic construct
EISEVQLVESGGGLVQPGGSLRLSCAASGFNFSYYSIHWVRQAPGKGLEWVASISSYYGYTSYADSVKGRFTISADTSKNTAYLQMNSLRAEDTAVYYCARYFLGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 178 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYRYPPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 4 aa, synthetic construct
SSSI

SEQ ID NO: 180 - protein, 10 aa, synthetic construct
SISSSYSSTY

SEQ ID NO: 181 - protein, 6 aa, synthetic construct
AWWWAL

SEQ ID NO: 182 - protein, 6 aa, synthetic construct
YVSSLI

SEQ ID NO: 183 - protein, 116 aa, synthetic construct
EISEVQLVESGGGLVQPGGSLRLSCAASGFNSSSIHWVRQAPGKGLEWVASISSSYSSTYYADSVKGRFTISADTSKNTAYLQMNSLRAEDTAVYYCARAWWWALDYWGQGTLVTV

SEQ ID NO: 184 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYVSSLITFGQGTKVEIKRT

SEQ ID NO: 185 - protein, 226 aa, synthetic construct
EISEVQLVESGGGLVQPGGSLRLSCAASGFNSSSIHWVRQAPGKGLEWVASISSSYSSTYYADSVKGRFTISADTSKNTAYLQMNSLRAEDTAVYYCARAWWWALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 186 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYVSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - protein, 10 aa, synthetic construct
SISPYYGYTS

SEQ ID NO: 188 - protein, 11 aa, synthetic construct
TYYWKYGGPAM

SEQ ID NO: 189 - protein, 7 aa, synthetic construct
WSWYSLI

SEQ ID NO: 190 - protein, 123 aa, synthetic construct
EISEVQLVESGGGLVQPGGSLRLSCAASGFNFSSSSIHWVRQAPGKGLEWVASISPYYGYTSYADSVKGRFTISADTSKNTAYLQMNSLRAEDTAVYYCARTYYWKYGGPAMDYWGQGTLVTV

SEQ ID NO: 191 - protein, 111 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQWSWYSLITFGQGTKVEIKRT

SEQ ID NO: 192 - protein, 233 aa, synthetic construct
EISEVQLVESGGGLVQPGGSLRLSCAASGFNFSSSSIHWVRQAPGKGLEWVASISPYYGYTSYADSVKGRFTISADTSKNTAYLQMNSLRAEDTAVYYCARTYYWKYGGPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 193 - protein, 216 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQWSWYSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 6 aa, synthetic construct
YKYMGM

SEQ ID NO: 195 - protein, 6 aa, synthetic construct
SSWMPI

SEQ ID NO: 196 - protein, 118 aa, synthetic construct
EISEVQLVESGGGLVQPGGSLRLSCAASGFNFYSSYIHWVRQAPGKGLEWVASIYSYSGYTSYADSVKGRFTISADTSKNTAYLQMNSLRAEDTAVYYCARYKYMGMDYWGQGTLVTV

SEQ ID NO: 197 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWMPITFGQGTKVEIKRT

SEQ ID NO: 198 - protein, 228 aa, synthetic construct
EISEVQLVESGGGLVQPGGSLRLSCAASGFNFYSSYIHWVRQAPGKGLEWVASIYSYSGYTSYADSVKGRFTISADTSKNTAYLQMNSLRAEDTAVYYCARYKYMGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 199 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWMPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 8 aa, synthetic construct
YWYYYNGM

SEQ ID NO: 201 - protein, 6 aa, synthetic construct
IRYVPI

SEQ ID NO: 202 - protein, 120 aa, synthetic construct
EISEVQLVESGGGLVQPGGSLRLSCAASGFNFYSSYIHWVRQAPGKGLEWVASIYSYSGYTSYADSVKGRFTISADTSKNTAYLQMNSLRAEDTAVYYCARYWYYYNGMDYWGQGTLVTV

SEQ ID NO: 203 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQIRYVPITFGQGTKVEIKRT

SEQ ID NO: 204 - protein, 230 aa, synthetic construct
EISEVQLVESGGGLVQPGGSLRLSCAASGFNFYSSYIHWVRQAPGKGLEWVASIYSYSGYTSYADSVKGRFTISADTSKNTAYLQMNSLRAEDTAVYYCARYWYYYNGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 205 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQIRYVPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 6 aa, synthetic construct
FSYSSI

SEQ ID NO: 207 - protein, 6 aa, synthetic construct
YYYFGM

SEQ ID NO: 208 - protein, 6 aa, synthetic construct
SWSMPL

SEQ ID NO: 209 - protein, 118 aa, synthetic construct
EISEVQLVESGGGLVQPGGSLRLSCAASGFNFSYSSIHWVRQAPGKGLEWVASIYPSYGYTSYADSVKGRFTISADTSKNTAYLQMNSLRAEDTAVYYCARYYYFGMDYWGQGTLVTV

SEQ ID NO: 210 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSMPLTFGQGTKVEIKRT

SEQ ID NO: 211 - protein, 228 aa, synthetic construct
EISEVQLVESGGGLVQPGGSLRLSCAASGFNFSYSSIHWVRQAPGKGLEWVASIYPSYGYTSYADSVKGRFTISADTSKNTAYLQMNSLRAEDTAVYYCARYYYFGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 212 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSMPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 6 aa, synthetic construct
VYYSSI

SEQ ID NO: 214 - protein, 6 aa, synthetic construct
WSYKAM

SEQ ID NO: 215 - protein, 6 aa, synthetic construct
DYYAPI

SEQ ID NO: 216 - protein, 118 aa, synthetic construct
EISEVQLVESGGGLVQPGGSLRLSCAASGFNVYYSSIHWVRQAPGKGLEWVASIYSYSGYTSYADSVKGRFTISADTSKNTAYLQMNSLRAEDTAVYYCARWSYKAMDYWGQGTLVTV

SEQ ID NO: 217 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQDYYAPITFGQGTKVEIKRT

SEQ ID NO: 218 - protein, 228 aa, synthetic construct
EISEVQLVESGGGLVQPGGSLRLSCAASGFNVYYSSIHWVRQAPGKGLEWVASIYSYSGYTSYADSVKGRFTISADTSKNTAYLQMNSLRAEDTAVYYCARWSYKAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 219 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQDYYAPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 6 aa, synthetic construct
VYSYSI

SEQ ID NO: 221 - protein, 10 aa, synthetic construct
SISSYSGYTS

SEQ ID NO: 222 - protein, 12 aa, synthetic construct
GSSWWSYYVKAM

SEQ ID NO: 223 - protein, 8 aa, synthetic construct
GDYRKFLI

SEQ ID NO: 224 - protein, 124 aa, synthetic construct
EISEVQLVESGGGLVQPGGSLRLSCAASGFNVYSYSIHWVRQAPGKGLEWVASISSYSGYTSYADSVKGRFTISADTSKNTAYLQMNSLRAEDTAVYYCARGSSWWSYYVKAMDYWGQGTLVTV

SEQ ID NO: 225 - protein, 112 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGDYRKFLITFGQGTKVEIKRT

SEQ ID NO: 226 - protein, 234 aa, synthetic construct
EISEVQLVESGGGLVQPGGSLRLSCAASGFNVYSYSIHWVRQAPGKGLEWVASISSYSGYTSYADSVKGRFTISADTSKNTAYLQMNSLRAEDTAVYYCARGSSWWSYYVKAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 227 - protein, 217 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGDYRKFLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 6 aa, synthetic construct
FYYYYI

SEQ ID NO: 229 - protein, 10 aa, synthetic construct
SISPYSGSTY

SEQ ID NO: 230 - protein, 11 aa, synthetic construct
QEWHYSYMYGM

SEQ ID NO: 231 - protein, 6 aa, synthetic construct
GSFSLI

SEQ ID NO: 232 - protein, 123 aa, synthetic construct
EISEVQLVESGGGLVQPGGSLRLSCAASGFNFYYYYIHWVRQAPGKGLEWVASISPYSGSTYYADSVKGRFTISADTSKNTAYLQMNSLRAEDTAVYYCARQEWHYSYMYGMDYWGQGTLVTV

SEQ ID NO: 233 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGSFSLITFGQGTKVEIKRT

SEQ ID NO: 234 - protein, 233 aa, synthetic construct
EISEVQLVESGGGLVQPGGSLRLSCAASGFNFYYYYIHWVRQAPGKGLEWVASISPYSGSTYYADSVKGRFTISADTSKNTAYLQMNSLRAEDTAVYYCARQEWHYSYMYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 235 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGSFSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 12 aa, synthetic construct
WHWGWGSYGWGM

SEQ ID NO: 237 - protein, 6 aa, synthetic construct
QWYVPI

SEQ ID NO: 238 - protein, 124 aa, synthetic construct
EISEVQLVESGGGLVQPGGSLRLSCAASGFNVYSSSIHWVRQAPGKGLEWVASIYPSYGYTSYADSVKGRFTISADTSKNTAYLQMNSLRAEDTAVYYCARWHWGWGSYGWGMDYWGQGTLVTV

SEQ ID NO: 239 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQQWYVPITFGQGTKVEIKRT

SEQ ID NO: 240 - protein, 234 aa, synthetic construct
EISEVQLVESGGGLVQPGGSLRLSCAASGFNVYSSSIHWVRQAPGKGLEWVASIYPSYGYTSYADSVKGRFTISADTSKNTAYLQMNSLRAEDTAVYYCARWHWGWGSYGWGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 241 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQQWYVPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 10 aa, synthetic construct
SISSYSGSTS

SEQ ID NO: 243 - protein, 12 aa, synthetic construct
DEFTYWGWSYAF

SEQ ID NO: 244 - protein, 6 aa, synthetic construct
SEYGLI

SEQ ID NO: 245 - protein, 124 aa, synthetic construct
EISEVQLVESGGGLVQPGGSLRLSCAASGFNFSYYSIHWVRQAPGKGLEWVASISSYSGSTSYADSVKGRFTISADTSKNTAYLQMNSLRAEDTAVYYCARDEFTYWGWSYAFDYWGQGTLVTV

SEQ ID NO: 246 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSEYGLITFGQGTKVEIKRT

SEQ ID NO: 247 - protein, 234 aa, synthetic construct
EISEVQLVESGGGLVQPGGSLRLSCAASGFNFSYYSIHWVRQAPGKGLEWVASISSYSGSTSYADSVKGRFTISADTSKNTAYLQMNSLRAEDTAVYYCARDEFTYWGWSYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 248 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSEYGLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 10 aa, synthetic construct
SISPYSGYTS

SEQ ID NO: 250 - protein, 13 aa, synthetic construct
GYGGWYWWYTFAF

SEQ ID NO: 251 - protein, 7 aa, synthetic construct
SSSSELL

SEQ ID NO: 252 - protein, 125 aa, synthetic construct
EISEVQLVESGGGLVQPGGSLRLSCAASGFNFSSSSIHWVRQAPGKGLEWVASISPYSGYTSYADSVKGRFTISADTSKNTAYLQMNSLRAEDTAVYYCARGYGGWYWWYTFAFDYWGQGTLVTV

SEQ ID NO: 253 - protein, 111 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSSSELLTFGQGTKVEIKRT

SEQ ID NO: 254 - protein, 235 aa, synthetic construct
EISEVQLVESGGGLVQPGGSLRLSCAASGFNFSSSSIHWVRQAPGKGLEWVASISPYSGYTSYADSVKGRFTISADTSKNTAYLQMNSLRAEDTAVYYCARGYGGWYWWYTFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 255 - protein, 216 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSSSELLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 13 aa, synthetic construct
SPWYVYWYFGSAL

SEQ ID NO: 257 - protein, 125 aa, synthetic construct
EISEVQLVESGGGLVQPGGSLRLSCAASGFNFSSSYIHWVRQAPGKGLEWVASISSSSGSTSYADSVKGRFTISADTSKNTAYLQMNSLRAEDTAVYYCARSPWYVYWYFGSALDYWGQGTLVTV

SEQ ID NO: 258 - protein, 235 aa, synthetic construct
EISEVQLVESGGGLVQPGGSLRLSCAASGFNFSSSYIHWVRQAPGKGLEWVASISSSSGSTSYADSVKGRFTISADTSKNTAYLQMNSLRAEDTAVYYCARSPWYVYWYFGSALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT